(19) United States
(12) Patent Application Publication
(10) Pub. No.: US 2008/0305044 A1
(43) Pub. Date: Dec. 11, 2008

(54) Engineered Antibodies and Immunoconjugates

(75) Inventors: Charlotte McDonagh, Bothell, WA (US); Paul Carter, Bothell, WA (US)

Correspondence Address: TOWNSEND AND TOWNSEND AND CREW LLP, TWO EMBARCADERO CENTER, 8TH FLOOR, SAN FRANCISCO, CA, 94111, US

(73) Assignee: Seattle Genetics, Inc., Bothell, WA (US)

(21) Appl. No.: 11/720,244

(22) PCT Filed: Nov. 29, 2005
(86) PCT No.: PCT/US2005/043257

§ 371 Date: Mar. 28, 2008

Related U.S. Application Data
(60) Provisional application No. 60/631,757, filed on Nov. 29, 2004.
(60) Provisional application No. 60/673,146, filed on Apr. 19, 2005.

(51) Int. Cl. A61K 49/00; C07K 16/00; C12N 9/00; C12P 21/04; A61P 35/04; A61K 39/44; C12N 5/02

(52) U.S. Cl. 424/9.1; 530/391.1; 530/391.3; 435/183; 424/178.1; 435/375; 435/69.6

(57) ABSTRACT

Antibody drug conjugates with predetermined sites and stoichiometries of drug attachment are provided. Also provided are methods of using antibody drug conjugates.

### CROSS REFERENCE TO RELATED APPLICATIONS

This application claims the benefit of U.S. Provisional Patent Application No. 60/631,757, filed Nov. 29, 2004, and of U.S. Provisional Patent Application No. 60/673,146, filed Apr. 19, 2005, each of which is hereby incorporated by reference herein in its entirety.

### BACKGROUND

The present invention is directed to engineered antibodies with predetermined points of attachment for an active moiety. In particular, the invention is directed to antibodies with predetermined points of attachment for active moieties by selective substitution of an amino acid residue(s) of the antibody.

The use of targeting monoclonal antibodies conjugated to radionuclides or other cytotoxic agents offers the possibility of delivering such agents directly to the tumor site, thereby limiting the exposure of normal tissues to the agents (see, e.g., Goldenberg, Semin. Nucl. Med. 19: 332 (1989)). In recent years, the potential of antibody-based therapy and its accuracy in the localization of tumor-associated antigens have been demonstrated both in the laboratory and clinical studies (see, e.g., Thorpe, TIBTECH 11:42 (1993); Goldenberg, Scientific American, Science & Medicine 1:64 (1994); Baldwin et al., U.S. Pat. Nos. 4,925,922 and 4,916,213; Young, U.S. Pat. Nos. 4,918,163 and 5,204,095; Irie et al., U.S. Pat. No. 5,196,337; Hellstrom et al., U.S. Pat. Nos. 5,134,075 and 5,171,665). In general, the use of radiolabeled antibodies or antibody fragments against tumor-associated markers has been more successful for localization of tumors than for therapy, in part because antibody uptake by the tumor is generally low, ranging from only 0.01% to 0.001% of the total dose injected (Vaughan et al., Brit. J. Radiol. 60:567 (1987)). Increasing the concentration of the radiolabel to increase the dosage to the tumor is generally counterproductive because this also increases exposure of healthy tissue to radioactivity.

Monoclonal antibodies can be conjugated to a variety of agents, other than radionuclides, to form immunoconjugates for use in diagnosis and therapy. These agents include chelates, which allow the immunoconjugate to form a stable bond with radioisotopes, and cytotoxic agents such as toxins and chemotherapy drugs. For example, cytotoxic agents that normally would be too toxic to patients if administered in a systemic fashion can be conjugated to anti-cancer antibodies in such a manner that their toxic effects become directed only to the tumor cells bearing the target antigens. The diagnostic or therapeutic efficacy of immunoconjugates depends upon several factors. Among these factors are the molar ratio of the agent to the antibody and the binding activity of the immunoconjugate.

Researchers have found that the maximum number of agents that can be directly linked to an antibody is limited by the number of modifiable sites on the antibody molecule and the potential loss of immunoreactivity of the antibody. For example, Kulkarni et al. (Cancer Research 41:2700-2706 (1981)) have reported that there is a limit to the number of drug molecules that can be incorporated into an antibody without significantly decreasing antigen-binding activity. Kulkarni et al. found that the highest incorporation obtained for methotrexate was about ten methotrexate molecules per-molecule of antibody, and that attempts to increase the drug-antibody molar ratio over about ten decreased the yield of immunoconjugate and damaged antibody activity. Kanellos et al. (JNC 75:319-329 (1985)) have reported similar results.

For monoclonal antibodies to function as the delivery vehicles for drugs and radionuclides, it is important to develop methods for their site-specific conjugations, with minimal perturbation of the resultant immunoreactivities. Most commonly, the conjugation of drugs and radionuclides is accomplished through covalent attachments to side chains of amino acid residues. Due to the non-site-restricted nature of these residues, it is difficult to avoid undesirable couplings at residues that lie within or are in close vicinity to the antigen binding site (ABS), leading to reduced affinity and heterogeneous antigen-binding properties. Alternatively, conjugation can be directed at sulfhydryl groups. However, direct labeling relies on the reduction of disulfide (S—S) bonds, with the possible risk of protein fragmentation. Incomplete reduction of such bonds can lead to heterogeneous patterns of attachment.

For example, early preclinical versions of the cAC10 antibody drug conjugate (directed to CD30) involved linkage of eight MMAE (monomethyl auristatin E) drug molecules to the antibody via the cysteine residues. The cysteine residues were obtained by reduction of the four interchain disulfide bonds (Doronina et al., Nat. Biotechnol. 21(7):778-84 (2003)). A recent report has described the effects of drug multiplicity on the in vivo parameters of cAC10 ADCs (Hamblett et al., Clin. Cancer Res. 15: 7063-7070 (2004)). cAC10 MMAE drug conjugates with 4 drug molecules attached per antibody (designated C8-E4, where C# indicates the number of interchain cysteine residues available for conjugation and E# indicates the average number of drug molecules attached per antibody molecule) have been shown to have a greater therapeutic window than cAC10 drug conjugates with 8 drugs attached per antibody (designated C8-E8) in animal models. C8-E4 displays similar pharmacokinetic properties to cAC11 alone, while C8-E8 is cleared from circulation more rapidly (Hamblett et al., supra). These characteristics suggest that C8-E4 may be a candidate for clinical development.

The preparation of C8-E4 from cAC10 may result in low yields and heterogeneity of drug attachment, depending on the method of conjugation. One method used to obtain MMAE conjugates with less than eight drugs loaded per antibody utilizes partial reduction of cysteine residues (Hamblett et al., supra). This conjugation process results in a mixture of species with zero, two, four, six or eight drug molecules per antibody molecule (designated C8-E0, C8-E2, C8-E4, C8-E6 and C8-E8, respectively), of which approximately 30% is C8-E4. This conjugate mixture can be separated by hydrophobic interaction chromatography to obtain pure C8-E4, but this process results in a further reduction in overall yield and remaining heterogeneity because the drugs are distributed over eight possible conjugation sites. Further, reduction of the heavy to light chain disulfide bond occurs at approximately double the frequency of the heavy to heavy disulfide bonds, resulting in a 2:1 ratio of the respective C8-E4 isomers. (See, e.g., Sun, et al., Bioconjug Chem 16:1282-1290 (2005).)

Thus, there is a need for antibodies having one or more predetermined sites for stoichiometric drug attachment. These and other limitations and problems of the past are solved by the present invention.

### BRIEF SUMMARY OF THE INVENTION

The invention relates to engineered antibodies and immunoconjugates. The invention provides engineered antibodies and immunoconjugates and methods of preparing such engineered antibodies and immunoconjugates. The invention also provides pharmaceutical compositions of immunoconjugates and methods of using immunoconjugates to treat or diagnose a variety of conditions and diseases.

In one aspect, the invention provides immunoconjugates including engineered antibodies having a functionally active antigen-binding site for a target antigen, at least one interchain cysteine residue, at least one amino acid substitution of an interchain cysteine residue, and a diagnostic, preventative or therapeutic agent conjugated to at least one interchain cysteine residue. In one embodiment, the invention provides immunoconjugates having four interchain cysteine residues and four amino acid substitutions of interchain cysteine residues. In a related embodiment, the invention provides immunoconjugates having two interchain cysteine residues and six amino acid substitutions of interchain cysteine residues. In another embodiment, the invention provides immunoconjugates that are of the IgG1 or IgG4 isotype. The amino acid substitutions can be, for example, cysteine to serine amino acid substitutions of the interchain cysteine residues.

In another aspect, the invention provides immunoconjugates as described above in which a therapeutic agent is conjugated to at least one interchain cysteine residue. In one embodiment, the therapeutic agent is an auristatin or auristatin derivative. In some embodiments, the auristatin derivative is dovaline-valine-dolaisoleunine-dolaproine-phenylalanine (MMAF) or monomethyauristatin E (MMAE).

In another aspect, the invention provides immunoconjugates as described above in which a diagnostic agent is conjugated to at least one interchain cysteine residue. The diagnostic agent can be, for example, a radioactive agent, an enzyme, a fluorescent compounds or an electron transfer agent.

In another aspect, the invention provides immunoconjugates as described above in which the antibody has a functionally active antigen-binding site for a target antigen. The antibody can bind to, for example, CD20, CD30, CD33, CD40, CD70 or Lewis Y. The antibody also can bind to an immunoglobulin gene superfamily member, a TNF receptor superfamily member, an integrin, a cytokine receptor, a chemokine receptor, a major histocompatibility protein, a lectin, or a complement control protein. In other examples, the antibody binds to a microbial antigen, or viral antigen. The antibody also can be an anti-nuclear antibody, anti-ds DNA antibody, anti-ss DNA antibody, anti-cardiolipin antibody IgM or IgG, anti-phospholipid antibody IgM or IgG, anti-SM antibody, anti-mitochondrial antibody, anti-thyroid antibody, anti-microsomal antibody, anti-thyroglobulin antibody, anti-SCL 70 antibody, anti-Jo antibody, anti-U1RNP antibody, anti-La/SSB antibody, anti-SSA antibody, anti-SSB antibody, anti-perital cells antibody, anti-histone antibody, anti-RNP antibody, anti-C ANCA antibody, anti-P ANCA antibody, anti-centromere antibody, anti-fibrillarin antibody, or anti-GBM antibody.

In another aspect, the invention provides immunoconjugates as described above in which the antibody is an antibody fragment. In one embodiment, the antibody fragment is Fab, Fab′ or scFvFc.

In another aspect, the invention provides immunoconjugates of the following formula:

Ab_{z}Aₐ-W_{w}-Y_{y}-D)ₚ
or a pharmaceutically acceptable salt or solvate thereof,
wherein:
Ab is an antibody,
A is a stretcher unit,
a is 0 or 1,
each W is independently a linker unit,
w is an integer ranging from 0 to 12,
Y is a spacer unit, and
y is 0, 1 or 2,
p ranges from 1 to about 20, and
D is a diagnostic, preventative and therapeutic agent, and
z is the number of predetermined conjugation sites on the protein.

In some embodiments, the immunoconjugates are of the formula: Ab-MC-vc-PAB-MMAF, Ab-MC-vc-PAB-MMAE, Ab-MC-MMAE or Ab-MC-MMAF.

In another aspect, the invention provides pharmaceutical compositions containing the immunoconjugates described above and a pharmaceutical acceptable carrier. In an embodiment, the immunoconjugate is formulated with a pharmaceutically acceptable parenteral vehicle. In another embodiment, the immunoconjugate is formulated in a unit dosage injectable form. In a related aspect, the invention provides an article of manufacture having an immunoconjugate conjugated to a therapeutic agent, a container, and a package insert or label indicating that the compound can be used to treat cancer characterized by the overexpression of at least one of CD20, CD30, CD33, CD40, CD70 and Lewis Y.

In another aspect, the invention provides methods of treating a variety of conditions or diseases using immunoconjugates described above that are conjugated to a therapeutic agent. In one embodiment, the methods involve killing or inhibiting the proliferation of tumor cells or cancer cells by treating tumor cells or cancer cells with an amount the immunoconjugate, or a pharmaceutically acceptable salt or solvate, effective to kill or inhibit the proliferation of the tumor cells or cancer cells. In another embodiment, the methods involve treating cancer by administering to a patient an amount of immunoconjugate, or a pharmaceutically acceptable salt or solvate, effective to treat cancer. In another embodiment, the methods involve treating an autoimmune disease by administering to a patient an amount of immunoconjugate, or a pharmaceutically acceptable salt or solvate, effective to treat the autoimmune disease. In yet another embodiment, the methods involve treating an infectious disease by administering to a patient an amount of an immunoconjugate, or a pharmaceutically acceptable salt or solvate, effective to treat the infectious disease.

In another aspect, the invention provides methods of diagnosing a variety of conditions or diseases using immunoconjugates described above that are conjugated to a diagnostic agent. In one embodiment, the methods involve diagnosing cancer by administering to a patient an effective amount of immunoconjugate that binds to an antigen overexpressed by the cancer, and detecting the immunoconjugate in the patient. In another embodiment, the methods involve diagnosing an infectious disease by administering to a patient an effective amount of the immunoconjugate that binds to a microbial or viral antigen, and detecting the immunoconjugate in the patient. In yet another embodiment, the methods involve diagnosing an autoimmune disease in a patient by administering an effective amount of immunoconjugate that binds to an antigen associated with the autoimmune disease, and detecting the immunoconjugate in the patient.

In another aspect, the invention provides methods of preparing an immunoconjugate involving culturing a host cell expressing an engineered antibody having a functionally active antigen-binding region for a target antigen, at least one interchain cysteine residue, and at least one amino acid substitution of an interchain cysteine residue. The host cell can be transformed or transfected with an isolated nucleic acid encoding the engineered antibody. The antibody can be recovered from the cultured host cells or the culture medium, and conjugated to a diagnostic, preventative or therapeutic agent via at least one interchain cysteine residue. In an embodiment, the antibody is an intact antibody or an antigen-binding fragment. In a preferred embodiment, the antigen binding fragment is an Fab, Fab′ or scFvFc.

The invention will best be understood by reference to the following detailed description of the preferred embodiment, taken in conjunction with the accompanying drawings. The discussion below is descriptive, illustrative and exemplary and is not to be taken as limiting the scope defined by any appended claims.

### BRIEF DESCRIPTION OF THE DRAWINGS

FIG. 1 shows the design and analysis of antibody Cys→Ser variants and corresponding antibody drug conjugates (ADCs). (A) Schematic representation of antibody variants and drug conjugates highlighting the location of accessible cysteines (diamonds), inter-chain disulfide bonds (−) and subsequently conjugated drugs (+). Antibodies and ADCs are identified by their variant name (see Table 1), and loading stoichiometry with the drug, MMAE. For example, C8-E8 denotes the ADC in which all eight solvent accessible interchain cysteine residues in the cAC10 parent antibody (C8) are conjugated to MMAE (E8). (B) SDS-PAGE analysis of antibody variants under non-reducing conditions. HHLL, HH, HL, H and L indicate migration patterns for antibody heavy-light chain tetramer, heavy chain dimer, heavy-light chain dimer, heavy chain and light chain, respectively. (C) SDS-PAGE analysis of antibody variant conjugates with MMAE under reducing conditions.

FIG. 2 shows titration profiles of a growth proliferation assay using antibody cysteine variants and parent cAC10 antibody conjugated to MC-vcMMAE. (A) Serial dilutions of cAC10 ADCs C2v1-E2, C4v1-E4, C4v2-E4, C6v1-E6 and C8-E4 were incubated with Karpas-299 cells for 96 hours. [H³]-TdR was then added and its incorporation measured. (B) Karpas-299 cells were incubated with cAC10 ADCs C2v1-E2, C2v2-E2 and C8-E2 for 96 hours. Resazurin was then added and dye reduction measured.

FIG. 3 shows single dose efficacy studies on SCID mice bearing Karpas-299 subcutaneous xenografts that were treated with antibody cysteine variants and parent cAC10 antibody conjugated to MC-vcMMAE. Mice were treated with a single dose of C2v1-E2 and C8-E2 at 2 mg/kg (A) and C4v1-E4, C4v2-E4, and C8-E4 at 1 mg/kg (B).

FIG. 4 shows plasmid map pBSSK AC10H.

FIG. 5 shows plasmid map pBSSK AC10 L.

FIG. 6 shows reverse phase HPLC analysis of ADCs under reducing conditions. (A) C8-E4M. (B) C8-E4. (C) C4v1-E4. (D) C4v2-E4. (See Table 1). Peaks were identified by the ratio of their absorbances at wavelengths of 248 nm and 280 nm. L-E0 and L-E1 are used to denote light chains loaded with 0 or 1 equivalents of MMAE, respectively, whereas H-E0, H-E1, H-E2 and H-E3 indicate heavy chains loaded with 0, 1, 2, or 3 equivalents of MMAE, respectively.

FIG. 7 shows single dose efficacy studies on SCID mice bearing L540cy subcutaneous xenografts. Mice were treated 12 days post tumor implant with a single dose of C2v1-E2, C2v2-E2 and C8-E2 at 6 mg/kg (A) or 12 mg/kg (B). Mice were dosed with C4v1-E4, C4v2-E4, C8-E4 and C8-E4M at 3 mg/kg (C) and 6 mg/kg (D).

### DEFINITIONS

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

Antibody. As used herein, “antibody” refers to monoclonal antibodies, such as murine, chimeric, human, or humanized antibodies, mixtures of antibodies, as well as antigen-binding fragments thereof. Such fragments include Fab, Fab′, F(ab)₂, and F(ab′)₂. Antibody fragments also include isolated fragments consisting of the light chain variable region, “Fv” fragments consisting of the variable regions of the heavy and light chains, and recombinant single chain polypeptide molecules in which light and heavy variable regions are connected by a peptide linker (e.g., scFv and scFvFc). In some embodiments, the antibody comprises at least one interchain cysteine residue.

Intact Antibody. An “intact” antibody is one which comprises a V_{L} and V_{H} antigen-binding variable regions as well as light chain constant domain (C_{L}) and heavy chain constant domains, C_{H}1, C_{H}2, C_{H}3, and C_{H}4. The constant domains may be native sequence constant domains (e.g., human native sequence constant domains) or amino acid sequence variants thereof.

Interchain Cysteine Residue: As used herein, “interchain cysteine residue” or “interchain cysteine” refer to a cysteine residue of an antibody chain that can be involved in the formation of an interchain disulfide bond with a cysteine residue of another chain of the unengineered antibody. The interchain cysteine residues are located in the C_{L} domain of the light chain, the C_{H}1 domain of the heavy chain, and in the hinge region. The number of interchain cysteine residues in an antibody can vary. For example, human IgG1, IgG2, IgG3 and IgG4 isotypes have 4, 6, 13 and 4 interchain cysteine bonds, respectively. In a specific example, by reference to antibody cAC10, the interchain cysteine thiols are located at amino acid position 214 of the light chain and at amino acid positions 220, 226 and 229 of the heavy chain, according to the numbering scheme of Kabat (Kabat et al., Sequences of Proteins of Immunological Interest, 5th ed. NIH, Bethesda, Md. (1991)).

Interchain Disulfide Bond. The term “interchain disulfide bond,” in the context of an antibody, refers to a disulfide bond between two heavy chains, or a heavy and a light chain.

Engineered Antibody. As used herein, an “engineered antibody” refers to a nonnaturally occurring intact antibody or antigen-binding fragment having at least one amino acid substitution of an interchain cysteine residue for another amino acid residue (e.g., a cysteine to serine substitution), and retaining at least one unsubstituted interchain cysteine residue.

Isomer. The term “isomer” in the context of an antibody refers to an antibody having a particular pattern or order of amino acid substitutions of interchain cysteine residues. In the context of an immunoconjugate, the term “isomer” refers to an antibody having a particular pattern or order of amino acid substitutions of interchain cysteine residues and/or a particular pattern of sites of conjugation of an active moiety or moieties. An isomer of an antibody can be referred to by the nomenclature C#v#, where C# indicates the number of interchain cysteine residues available for conjugation and v# refers to a particular pattern or order of interchain cysteine residues. An isomer of an immunoconjugate can be referred to by the nomenclature C#v#-Y, where C# and v# have the same meaning as stated above and Y refers to the average number of diagnostic, preventative or therapeutic agents attached per antibody molecule.

Fully-Loaded. The term “fully-loaded” refers to an antibody in which the predetermined points of conjugation of a particular type and/or of similar reactivity are conjugated to an active moiety, resulting in a homogeneous population of the immunoconjugate (C#=Y).

Partially-Loaded. The term “partially-loaded” refers to an antibody in which only some of the predetermined points of conjugation of a particular type and/or of a similar reactivity are conjugated to an active moiety, resulting in formation of a certain isomer or isomers of the immunoconjugate (C#>Y).

Diagnostic, Preventative or Therapeutic Agent. As used herein, a “diagnostic, preventative or therapeutic agent” is an active moiety such as a macromolecule, molecule or atom which is conjugated to an antibody to produce an immunoconjugate which is useful for diagnosis, prevention and/or for therapy. Examples of diagnostic, preventative or therapeutic agents include drugs, toxins, and detectable labels.

Immunoconjugate. As used herein, an “immunoconjugate” is a molecule comprising an antibody conjugated directly or indirectly to at least one diagnostic, preventative and/or therapeutic agent, or a chelating agent that binds the diagnostic, preventative and/or therapeutic agent. An immunoconjugate retains the immunoreactivity of the antibody, e.g., the antibody has approximately the same, or only slightly reduced, ability to bind the antigen after conjugation as before conjugation. As used herein, an immunoconjugate is also referred to as an antibody drug conjugate (ADC).

Functionally Active. The term “functionally active,” in the context of an antibody means the antibody immunospecifically binds to a target antigen.

Isolated. The term “isolated,” in the context of a molecule or macromolecule (e.g., an antibody or nucleic acid) is one which has been identified and separated and/or recovered from a component of its natural environment. Contaminant components of its natural environment are materials which would interfere with the desired use (e.g., diagnostic or therapeutic) of the molecule, and may include enzymes, hormones, and other proteinaceous or nonproteinaceous solutes. In some embodiments, an isolated molecule or macromolecule will be purified (1) to greater than 95%, or greater than 99%, by weight of the molecule or macromolecule as determined by, for example, the Lowry or Bradford methods, (2) to a degree sufficient to obtain at least 15 residues of N-terminal or internal amino acid sequence by use of a spinning cup sequenator, or (3) to homogeneity by SDS-PAGE under reducing or nonreducing conditions determined by, for example, Coomassie blue or, preferably, silver staining methods. Isolated molecules and macromolecules include the molecule and macromolecule in situ within recombinant cells since at least one component of the molecules' and macromolecules' natural environment will not be present. Ordinarily, however, isolated molecules and macromolecules will be prepared by at least one purification step.

Structural gene. As used herein, a “structural gene” is a DNA molecule having a sequence that is transcribed into messenger RNA (mRNA) which is then translated into a sequence of amino acids characteristic of a specific polypeptide.

Promoter. As used herein, a “promoter” is a sequence of a nucleic acid that directs the transcription of a structural gene to produce mRNA. Typically, a promoter is located in the 5′ region of a gene, proximal to the start codon of a structural gene. If a promoter is an inducible promoter, then the rate of transcription increases in response to an inducing agent. In contrast, the rate of transcription is not regulated by an inducing agent if the promoter is a constitutive promoter.

Enhancer. As used herein, an “enhancer” is a promoter element that can increase the efficiency with which a particular gene is transcribed into mRNA, irrespective of the distance or orientation of the enhancer relative to the start site of transcription.

Complementary DNA (cDNA). As used herein, “complementary DNA” is a single-stranded DNA molecule that is formed from an mRNA template by the enzyme reverse transcriptase. Typically, a primer complementary to a portion(s) of mRNA is employed for the initiation of reverse transcription. Those skilled in the art also use the term “cDNA” to refer to a double-stranded DNA molecule consisting of such a single-stranded DNA molecule and its complement.

Expression. As used herein, “expression” is the process by which a polypeptide is produced from a structural gene or cDNA molecule. The process involves transcription of the coding region into mRNA and the translation of the mRNA into a polypeptide(s).

Cloning vector. As used herein, a “cloning vector” is a DNA molecule, such as a plasmid, cosmid, or bacteriophage, which has the capability of replicating autonomously in a host cell and which is used to transform cells for gene manipulation. Cloning vectors typically contain one or a small number of restriction endonuclease recognition sites at which foreign DNA sequences may be inserted in a determinable fashion without loss of an essential biological function of the vector, as well as a marker gene which is suitable for use in the identification and selection of cells transformed with the cloning vector. Marker genes typically include genes that provide tetracycline resistance or ampicillin resistance.

Expression vector. As used herein, an “expression vector” is a DNA molecule comprising a heterologous structural gene or cDNA encoding a foreign protein which provides for the expression of the foreign protein in a recombinant host. Typically, the expression of the heterologous gene is placed under the control of (i.e., operably linked to) certain regulatory sequences such as promoter and/or enhancer sequences. Promoter sequences may be either constitutive or inducible.

Recombinant Host. A “recombinant host” may be any prokaryotic or eukaryotic cell for expression of a heterologous (foreign) protein. In some embodiments, the recombinant host contains a cloning vector or an expression vector. This term is also meant to include those prokaryotic or eukaryotic cells that have been genetically engineered to contain a nucleic acid encoding the heterologous protein in the chromosome or genome of the host cell. For examples of suitable hosts, see, e.g., Sambrook et al., Molecular Cloning: A Laboratory Manual, Second Edition, Cold Spring Harbor Laboratory, Cold Spring Harbor, N.Y. (1989); Sambrook et al., Molecular Cloning, A Laboratory Manual, Third Edition, Cold Spring Harbor Publish., Cold Spring Harbor, N.Y. (2001); and Ausubel et al., Current Protocols in Molecular Biology, 4th ed., John Wiley and Sons, New York (1999); all of which are incorporated by reference herein.

MMAE. The abbreviation “MMAE” refers to monomethyl auristatin E:

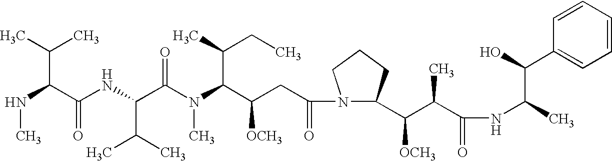

MMAF. The abbreviation “MMAF” refers to dovaline-valine-dolaisoleucine-dolaproline-phenylalanine:

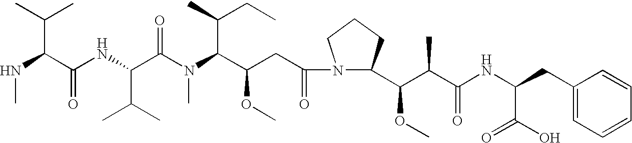

AFP. The abbreviation “AFP” refers to dimethylvaline-valine-dolaisoleucine-dolaproline-phenylalanine-p-phenylenediamine:

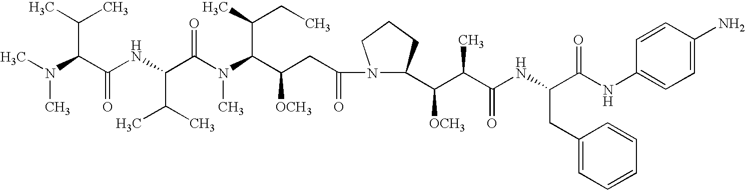

AEB. The abbreviation “AEB” refers to an ester produced by reacting auristatin E with paraacetyl benzoic acid.

AEVB. The abbreviation “AEVB” refers to an ester produced by reacting auristatin E with benzoylvaleric acid.

Patient. A “patient” includes, but is not limited to, a human, rat, mouse, guinea pig, monkey, pig, goat, cow, horse, dog, cat, bird and fowl.

Effective Amount. The term “effective amount” refers to an amount of a diagnostic, preventative or therapeutic agent sufficient for diagnosis, prevention or treatment of a disease or disorder in a mammal.

Therapeutically Effective Amount. The term “therapeutically effective amount” refers to an amount of a drug, toxin or other molecule effective to prevent or treat a disease or disorder in a mammal. In the case of cancer, the therapeutically effective amount may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug, toxin or other molecule may prevent growth and/or kill existing cancer cells, it may be cytostatic and/or cytotoxic. For cancer therapy, efficacy can, for example, be measured by assessing the time to disease progression (TTP) and/or determining the response rate (RR).

The phrase “pharmaceutically acceptable salt,” as used herein, refers to pharmaceutically acceptable organic or inorganic salts of a molecule or macromolecule. Acid addition salts can be formed with amino groups. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′ methylene bis-(2-hydroxy 3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Where multiple charged atoms are part of the pharmaceutically acceptable salt, the salt can have multiple counter ions. Hence, a pharmaceutically acceptable salt can have one or more charged atoms and/or one or more counterion.

“Pharmaceutically acceptable solvate” or “solvate” refer to an association of one or more solvent molecules and a molecule or macromolecule. Examples of solvents that form pharmaceutically acceptable solvates include, but are not limited to, water, isopropanol, ethanol, methanol, DMSO, ethyl acetate, acetic acid, and ethanolamine.

### DETAILED DESCRIPTION

The present invention provides engineered antibodies and immunoconjugates, and methods of preparing such antibodies and immunoconjugates. The engineered antibodies have at least one predetermined site for conjugation to an active moiety, such as a diagnostic, preventative or therapeutic agent. In some aspects, the engineered antibodies can be stoichiometrically conjugated to a diagnostic, preventative or therapeutic agent to form immunoconjugates with predetermined average loading of the agent. The immunoconjugates can be used therapeutically, diagnostically (e.g., in vitro or in vivo), for in vivo imaging, and for other uses. For clarity of disclosure, and not by way of limitation, the detailed description of the invention is divided into the subsections which follow.

### Engineered Antibodies

In one aspect, engineered antibodies are provided. An engineered antibody has an amino acid substitution of at least one interchain cysteine residue, while retaining at least one interchain cysteine residue for conjugation to a diagnostic, preventative or therapeutic agent.

In some embodiments, the antibody is an intact antibody. The antibody can be, for example, of the IgG, IgA, IgM, IgD or IgE class, and within these classes, various subclasses, such as an IgG1, IgG2, IgG3, IgG4, IgA1 or IgA2 isotypes. For example, in some embodiments, the antibody can be an IgG, such as an IgG1, IgG2, IgG3 or IgG4.

In some embodiments, the engineered antibody comprises at least one amino acid substitution replacing an interchain cysteine residue with another amino acid. The interchain cysteine residue can be involved in the formation of an interchain disulfide bond between light and heavy chains and/or between heavy chains. Thus, the amino acid substitution can be in the interchain cysteine residues in the C_{L} domain of the light chain, the C_{H}1 domain of the heavy chain, and/or in the hinge region. For example, with reference to antibody cAC10, the interchain cysteine residues are at amino acid positions 214 of the light chain and at amino acid positions 220 (C_{H}1) and 226 and 229 (hinge region) in the heavy chain in the numbering scheme of Kabat (Kabat et al., Sequences of Proteins of Immunological Interest, 5th ed. NIH, Bethesda, Md. (1991)). One or more of these interchain cysteine residues in cAC10 can be substituted.

In some embodiments, the amino acid substitution is a serine for a cysteine residue. In some embodiments, the amino acid substitution introduces is a serine or threonine residue. In some embodiments, the amino acid substitution introduces is a serine, threonine or glycine residue. In some embodiments, the amino acid substitution introduces a neutral (e.g., serine, threonine or glycine) or hydrophilic (e.g., methionine, alanine, valine, leucine or isoleucine) residue. In some embodiments, the amino acid substitution introduces a natural amino acid, other than a cysteine residue.

The engineered antibody retains at least one unsubstituted interchain cysteine residue for conjugation to an active moiety. The number of retained intercysteine residues in an engineered antibody is greater than zero but less than the total number of interchain cysteine residues in the parent (non-engineered) antibody. Thus, in some embodiments, the engineered antibody has at least one, at least two, at least three, at least four, at least five, at least six or at least seven interchain cysteine residues. In typical embodiments, the engineered antibody has an even integral number of interchain cysteine residues (e.g., at least two, four, six or eight reactive sites). In some embodiments, the engineered antibody has less than eight interchain cysteine residues.

In a typical embodiment, the interchain cysteine residues are substituted in a pairwise manner, in which both cysteine residues involved in the formation of an interchain disulfide bond are substituted. (Such interchain cysteine residues can be referred to as “complementary” interchain cysteine residues.) For example, if the C_{L} interchain cysteine residue(s) are substituted, the complementary C_{H}1 interchain cysteine residue(s) might also be substituted. In another example, each pair of the interchain cysteine residues in the hinge region can be substituted or remain unsubstituted in a pairwise manner. In other embodiments, an interchain cysteine residue can be substituted while the complementary residue can remain unsubstituted.

In some embodiments, the engineered antibody comprises light chains each having an amino acid substitution of the C_{L} interchain cysteine residue and heavy chains each having an amino acid substitution of the C_{H}1 interchain cysteine residue and retaining the interchain cysteine residues in the hinge region. In a related embodiment, an immunoconjugate of the engineered antibody has active moieties conjugated to the interchain cysteine residues of the hinge region.

In some embodiments, the engineered antibody comprises light chains each having an amino acid substitution of the C_{L} interchain cysteine residue and heavy chains each having an amino acid substitution of the C_{H}1 interchain cysteine residue and an amino acid substitution of at least one of the interchain cysteine residues in the hinge region. In a related embodiment, an immunoconjugate of the engineered antibody has active moieties conjugated to the remaining interchain cysteine residues of the hinge region.

In some embodiments, the engineered antibody comprises light chains each having the C_{L} interchain cysteine residue and heavy chains each retaining the C_{H}1 interchain cysteine residue and having amino acid substitutions of the hinge region interchain cysteine residues. In a related embodiment, an immunoconjugate of such an engineered antibody has active moieties conjugated to the C_{L} interchain cysteine residues and heavy chains C_{H}1 interchain cysteine residues.

In some embodiments, the engineered antibody comprises light chains each having the C_{L} interchain cysteine residue and heavy chains each retaining the C_{H}1 interchain cysteine residue and having amino acid substitutions of at least one but less than all of the hinge region interchain cysteine residues. In a related embodiment, an immunoconjugate of such an engineered antibody has active moieties conjugated to the C_{L} interchain cysteine residues, to heavy chains C_{H}1 interchain cysteine residues and to the remaining interchain cysteine residues.

In some embodiments, the engineered antibody comprises light chains each having the C_{L} interchain cysteine residue and heavy chains each having an amino acid substitution of the C_{H}1 interchain cysteine residue and an amino acid substitution of at least one of the hinge region interchain cysteine residues. In a related embodiment, an immunoconjugate of the engineered antibody has active moieties conjugated to the C_{L} interchain cysteines and to the remaining interchain cysteine residues of the hinge region.

In some embodiments, the engineered antibody comprises light chains each having the C_{L} interchain cysteine residue and heavy chains each having an amino acid substitution of the C_{H}1 interchain cysteine residue and an amino acid substitution of the hinge region interchain cysteine residues. In a related embodiment, an immunoconjugate of the engineered antibody has active moieties conjugated to the C_{L} interchain cysteine residues.

In some embodiments, the engineered antibody comprises light chains each having an amino acid substitution of the C_{L} interchain cysteine residue and heavy chains each having the C_{H}1 interchain cysteine residue and the hinge region interchain cysteine residues. In a related embodiment, an immunoconjugate of the engineered antibody has active moieties conjugated to the C_{H}1 interchain cysteine residues and to the interchain cysteine residues of the hinge region.

In some embodiments, the engineered antibody comprises light chains each having an amino acid substitution of the C_{L} interchain cysteine residue and heavy chains each having the C_{H}1 interchain cysteine residue and having an amino acid substitution of at least one of the hinge region interchain cysteine residues. In a related embodiment, an immunoconjugate of the engineered antibody has active moieties conjugated to the C_{H}1 interchain cysteine residues and to the remaining interchain cysteine residues of the hinge region.

In some embodiments, the engineered antibody comprises light chains each having an amino acid substitution of the C_{L} interchain cysteine residue and heavy chains each having the C_{H}1 interchain cysteine residue and having an amino acid substitution of the hinge region interchain cysteine residues. In a related embodiment, an immunoconjugate of the engineered antibody has active moieties conjugated to the C_{H}1 interchain cysteine residues.

In an exemplary embodiment where the parent antibody has eight interchain cysteine residues, the engineered antibody comprises light chains each having an amino acid substitution of the C_{L} interchain cysteine residue and heavy chains each having an amino acid substitution of the C_{H}1 interchain cysteine residue and retaining the interchain cysteine residues in the hinge region. In a related embodiment, an immunoconjugate of the engineered antibody has four active moieties conjugated to the interchain cysteine residues of the hinge region.

In an exemplary embodiment where the parent antibody has eight interchain cysteine residues, the engineered antibody comprises light chains each having the C_{L} interchain cysteine residue and heavy chains each retaining the C_{H}1 interchain cysteine residue and having amino acid substitutions of both hinge region interchain cysteine residues. In a related embodiment, an immunoconjugate of such an engineered antibody has four active moieties conjugated to the C_{L} interchain cysteine residues and heavy chains C_{H}1 interchain cysteine residues.

In an exemplary embodiment where the parent antibody has eight interchain cysteine residues, the engineered antibody comprises light chains each having the C_{L} interchain cysteine residue and heavy chains each having an amino acid substitution of the C_{H}1 interchain cysteine residue and an amino acid substitution of one of the hinge region interchain cysteine residues. In a related embodiment, an immunoconjugate of the engineered antibody has four active moieties conjugated to the C_{L} interchain cysteines and to the remaining interchain cysteine residues of the hinge region.

In an exemplary embodiment where the parent antibody has eight interchain cysteine residues, the engineered antibody comprises light chains each having an amino acid substitution of the C_{L} interchain cysteine residue and heavy chains each having an amino acid substitution of the C_{H}1 interchain cysteine residue and a substitution of one of the hinge region interchain cysteine residues. In a related embodiment, an immunoconjugate of the engineered antibody has two active moieties conjugated to the remaining interchain cysteine residues of the hinge region.

In an exemplary embodiment where the parent antibody has eight interchain cysteine residues, the engineered antibody comprises light chains each having the C_{L} interchain cysteine residue and heavy chains each having an amino acid substitution of the C_{H}1 interchain cysteine residue and an amino acid substitution of both hinge region interchain cysteine residues. In a related embodiment, an immunoconjugate of the engineered antibody has two active moieties conjugated to the remaining interchain cysteine residues of the hinge region.

In an exemplary embodiment where the parent antibody has eight interchain cysteine residues, the engineered antibody comprises light chains each having the C_{L} interchain cysteine residue and heavy chains each having the C_{H}1 interchain cysteine residue and an amino acid substitution of one of the hinge region interchain cysteine residues. In a related embodiment, an immunoconjugate of the engineered antibody has six active moieties conjugated to the C_{L} interchain cysteine residues and to the remaining interchain cysteine residues of the hinge region.

In an exemplary embodiment where the parent antibody has eight interchain cysteine residues, the engineered antibody comprises light chains each having the C_{L} interchain cysteine residue and heavy chains each having an amino acid substitution of the C_{H}1 interchain cysteine residue and retaining both of the hinge region interchain cysteine residues. In a related embodiment, an immunoconjugate of the engineered antibody has six active moieties conjugated to the C_{L} interchain cysteine residues and to the interchain cysteine residues of the hinge region.

In an exemplary embodiment where the parent antibody has eight interchain cysteine residues, the engineered antibody comprises light chains each having an amino acid substitution of the C_{L} interchain cysteine residue and heavy chains each retaining the C_{H}1 interchain cysteine residue and both of the hinge region interchain cysteine residues. In a related embodiment, an immunoconjugate of the engineered antibody has six active moieties conjugated to the C_{H}1 interchain cysteine residues and to the interchain cysteine residues of the hinge region.

The antibody also can be an antigen-binding antibody fragment such as, for example, a Fab, a F(ab′), a F(ab′)₂, a Fd chain, a single-chain Fv (e.g., scFv and scFvFc), a single-chain antibody, a disulfide-linked Fv (sdFv), a fragment comprising either a V_{L} or V_{H} domain, a minibody, a maxibody, an F(ab′)₃, or fragments produced by a Fab expression library. Antigen-binding antibody fragments, including single-chain antibodies, can comprise the variable region(s) alone or in combination with the entirety or a portion of the following: hinge region, C_{H}1, C_{H}2, C_{H}3, C_{H}4 and/or C_{L} domains. Also, antigen-binding fragments can comprise any combination of variable region(s) with a hinge region, C_{H}1, C_{H}2, C_{H}3, C_{H}4 and/or C_{L} domains. See also Holliger and Hudson, Nat. Biotechnol. 23:1126-1136 (2005), the disclosure of which is incorporated by reference herein.

In some embodiments, an antibody fragment comprises at least one domain, or part of a domain, that includes at least one interchain cysteine residue. For example, the antibody fragment can include a hinge region, a C_{L} and C_{H}1 domains, C_{L} and C_{H}1 domains and a hinge region, or the like.

The antibody fragment can be of any suitable antibody class (e.g., IgG, IgA, IgM, IgD and IgE) and subclass (e.g., IgG1, IgG2, IgG3, IgG4, IgA1 and IgA2).

Typically, the antibodies are human, rodent (e.g., mouse, rat or hamster), donkey, sheep, rabbit, goat, guinea pig, camelid, horse, or chicken. As used herein, “human” antibodies include antibodies having the amino acid sequence of a human immunoglobulin and include antibodies isolated from human immunoglobulin libraries, from human B cells, or from animals transgenic for one or more human immunoglobulins, as described infra and, for example in Reichert et al. (Nat. Biotechnol. 23:1073-8 (2005)) and in U.S. Pat. Nos. 5,939,598 and 6,111,166. The antibodies may be monospecific, bispecific, trispecific, or of greater multispecificity.

The antibody is typically a monoclonal antibody but also can be a mixture of monoclonal antibodies. When the subject is a human subject, the antibody may be obtained by immunizing any animal capable of mounting a usable immune response to the antigen. The animal may be a mouse, rat, goat, sheep, rabbit or other suitable experimental animal. The antigen may be presented in the form of a naturally occurring immunogen, or a synthetic immunogenic conjugate of a hapten and an immunogenic carrier. The antibody producing cells of the immunized animal may be fused with “immortal” or “immortalized” human or animal cells to obtain a hybridoma which produces the antibody. If desired, the genes encoding one or more of the immunoglobulin chains may be cloned so that the antibody may be produced in different host cells, and if desired, the genes may be mutated so as to alter the sequence and hence the immunological characteristics of the antibody produced. (See also Teng et al. Proc. Natl. Acad. Sci. USA. 80:7308-7312 (1983); Kozbor et al., Immunology Today 4:72-79 (1983); and Olsson et al., Meth. Enzymol. 92:3-16 (1982)). Human monoclonal antibodies may be made by any of numerous techniques known in the art, such as phage display (see, e.g., Hoogenboom, Nat. Biotechnol. 23:1105-16 (2005); transgenic mice expressing human immunoglobulin genes (see, e.g., Lonberg, Nat. Biotechnol. 23:1117-25 (2005)); ribosome-, mRNA- and yeast-display libraries (see, e.g., Hoogenboom, supra), and human hybridomas from patients (Brändlein et al., Histol. Histopathol. 19:897-905 (2004); and Illert et al., Oncol. Rep. 13:765-70 (2005)), and/or single-antigen selected lymphocytes (see, e.g., Lagerkvist et al., Biotechniques 18:862-9 (1995); and Babcook et al., Proc. Natl. Acad. Sci. USA 93:7843-8 (1996)).

The antibody can be, for example, a murine, a chimeric, humanized, or fully human antibody produced by techniques well-known to one of skill in the art. Recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as those having a variable region derived from a murine monoclonal and human immunoglobulin constant regions. (See, e.g., Cabilly et al., U.S. Pat. No. 4,816,567; and Boss et al., U.S. Pat. No. 4,816,397, which are incorporated herein by reference in their entirety.) In some embodiments, the antibody light chain constant region domain is not chimeric. In some embodiments, the antibody heavy chain constant region is not chimeric. In this context, “chimeric” refers to a constant region or constant region domain composed of portions from two different species.

The antibody can also be a bispecific antibody. Methods for making bispecific antibodies are known in the art. Traditional production of full-length bispecific antibodies is based on the coexpression of two immunoglobulin heavy chain-light chain pairs, where the two chains have different specificities (Milstein et al., Nature 305:537-539 (1983)). For further details for generating bispecific antibodies see, for example, Suresh et al., Methods in Enzymology 121:210 (1986); Rodrigues et al., J. Immunology 151:6954-6961 (1993); Carter et al., Bio/Technology 10:163-167 (1992); Carter et al., J. Hematotherapy 4:463-470 (1995); Merchant et al., Nature Biotechnology 16:677-681 (1998)). Using such techniques, bispecific antibodies can be prepared for use in the treatment or prevention of disease. Bifunctional antibodies are also described in European Patent Publication No. EPA 0 105 360. Hybrid or bifunctional antibodies can be derived either biologically, i.e., by cell fusion techniques, or chemically, especially with cross-linking agents or disulfide-bridge forming reagents, and may comprise whole antibodies or fragments thereof. Methods for obtaining such hybrid antibodies are disclosed for example, in International Publication WO 83/03679, and European Patent Publication No. EPA 0 217 577, both of which are incorporated herein by reference.

In some embodiments, the antibody constant domains have effector function. The term “antibody effector function(s),” or AEC, as used herein refers to a function contributed by an Fc domain(s) of an Ig. Such function can be effected by, for example, binding of an Fc effector domain(s) to an Fc receptor on an immune cell with phagocytic or lytic activity or by binding of an Fc effector domain(s) to components of the complement system. The effector function can be, for example, “antibody-dependent cellular cytotoxicity” or ADCC, “antibody-dependent cellular phagocytosis” or ADCP, “complement-dependent cytotoxicity” or CDC. In other embodiments, the constant domain(s) lacks one or more effector functions.

The antibodies may be directed against an antigen of interest, such as diagnostic preventative and/or therapeutic interest. For example, the antigen can be one associated with infectious pathogens (such as but not limited to viruses, bacteria, fungi, and protozoa), parasites, tumor cells, or particular medical conditions. In the case of a tumor-associated antigen (TAA), the cancer may be of the immune system, lung, colon, rectum, breast, ovary, prostate gland, head, neck, bone, or any other anatomical location. In some embodiments, the antigen is CD2, CD20, CD22, CD30, CD33, CD38, CD40, CD52, CD70, HER2, EGFR, VEGF, CEA, HLA-DR, HLA-Dr10, CA125, CA15-3, CA19-9, L6, Lewis X, Lewis Y, alpha fetoprotein, CA 242, placental alkaline phosphatase, prostate specific membrane antigen, prostate specific antigen, prostatic acid phosphatase, epidermal growth factor, MAGE-1, MAGE-2, MAGE-3, MAGE-4, anti-transferrin receptor, p97, MUC1, gp100, MART1, IL-2 receptor, human chorionic gonadotropin, mucin, P21, MPG, and Neu oncogene product.

Some specific useful antibodies include, but are not limited to, BR96 mAb (Trail et al., Science 261:212-215 (1993)), BR64 (Trail et al., Cancer Research 57:100-105 (1997)), mAbs against the CD 40 antigen, such as S2C6 mAb (Francisco et al., Cancer Res. 60:3225-3231 (2000)), and mAbs against the CD30 antigen, such as AC10 (Bowen et al., J. Immunol. 151:5896-5906 (1993)). Many other internalizing antibodies that bind to tumor specific antigens can be used, and have been reviewed (see, e.g., Franke et al., Cancer Biother. Radiopharm. 15:459-76 (2000); Murray, Semin Oncol. 27:64-70 (2000); Breitling et al., Recombinant Antibodies, John Wiley, and Sons, New York, 1998). The disclosures of these references are incorporated by reference herein.

In some embodiments, the antigen is a “tumor-specific antigen.” A “tumor-specific antigen” as used herein refers to an antigen characteristic of a particular tumor, or strongly correlated with such a tumor. However, tumor-specific antigens are not necessarily unique to tumor tissue, i.e., antibodies to tumor-specific antigens may cross-react with antigens of normal tissue. Where a tumor-specific antigen is not unique to tumor cells, it frequently occurs that, as a practical matter, antibodies binding to tumor-specific antigens are sufficiently specific to tumor cells to carry out the desired procedures without unwarranted risk or interference due to cross-reactions. Many factors contribute to this practical specificity. For example, the amount of antigen on the tumor cell may greatly exceed the amount of the cross-reactive antigen found on normal cells, or the antigen on the tumor cells may be more effectively presented. Therefore the term “tumor-specific antigen” relates herein to a specificity of practical utility, and is not intended to denote absolute specificity or to imply an antigen is unique to the tumor.

The nucleotide sequence encoding antibodies that are immunospecific for tumor associated or tumor specific antigens can be obtained, e.g., from the GenBank database or a database like it, commercial sources, literature publications, or by routine cloning and sequencing.

In some embodiments, the antibodies are directed against an antigen for the diagnosis, treatment or prevention of an autoimmune disease. Antibodies immunospecific for an antigen of a cell that is responsible for producing autoimmune antibodies can be obtained from the GenBank database or a database like it, a commercial or other source or produced by any method known to one of skill in the art such as, e.g., chemical synthesis or recombinant expression techniques.

In some embodiments, the antibody is an anti-nuclear antibody; anti-ds DNA; anti-ss DNA, anti-cardiolipin antibody IgM, IgG; anti-phospholipid antibody IgM, IgG; anti-SM antibody; anti-mitochondrial antibody; thyroid antibody; microsomal antibody; thyroglobulin antibody; anti-SCL 70; anti-Jo; anti-U1RNP; anti-La/SSB; anti-SSA; anti-SSB; anti-perital cells antibody; anti-histones; anti-RNP; anti-C ANCA; anti-P ANCA; anti-centromere; anti-fibrillarin, or an anti-GBM antibody.

In some embodiments, the antibody can bind to a receptor or a receptor complex expressed on a target cell (e.g., an activated lymphocyte). The receptor or receptor complex can comprise an immunoglobulin gene superfamily member, a TNF receptor superfamily member, an integrin, a cytokine receptor, a chemokine receptor, a major histocompatibility protein, a lectin, or a complement control protein. Non-limiting examples of suitable immunoglobulin superfamily members are CD2, CD3, CD4, CD8, CD19, CD22, CD28, CD79, CD90, CD152/CTLA 4, PD 1, and ICOS. Non-limiting examples of suitable TNF receptor superfamily members are CD27, CD40, CD95/Fas, CD134/OX40, CD137/4 1BB, TNF R1, TNF R2, RANK, TACI, BCMA, osteoprotegerin, Apo2/TRAIL R1, TRAIL R2, TRAIL R3, TRAIL R4, and APO 3. Non-limiting examples of suitable integrins are CD11a, CD11b, CD11c, CD18, CD29, CD41, CD49a, CD49b, CD49c, CD49d, CD49e, CD49f, CD103, and CD104. Non-limiting examples of suitable lectins are C type, S type, and I type lectin. In other embodiments, the receptor is CD70.

In some embodiments, the antibody is immunospecific for a viral or a microbial antigen. As used herein, the term “viral antigen” includes, but is not limited to, any viral peptide, polypeptide protein (e.g., HIV gp120, HIV nef, RSV F glycoprotein, influenza virus neuraminidase, influenza virus hemagglutinin, HTLV tax, herpes simplex virus glycoprotein (e.g., gB, gC, gD, and gE) and hepatitis B surface antigen) that is capable of eliciting an immune response. As used herein, the term “microbial antigen” includes, but is not limited to, any microbial peptide, polypeptide, protein, saccharide, polysaccharide, or lipid molecule (e.g., a bacterial, fungi, pathogenic protozoa, or yeast polypeptide including, e.g., LPS and capsular polysaccharide 5/8) that is capable of eliciting an immune response.

Antibodies immunospecific for a viral or microbial antigen can be obtained commercially, for example, from BD Biosciences (San Francisco, Calif.), Chemicon International, Inc. (Temecula, Calif.), or Vector Laboratories, Inc. (Burlingame, Calif.) or produced by any method known to one of skill in the art such as, e.g., chemical synthesis or recombinant expression techniques. The nucleotide sequence encoding antibodies that are immunospecific for a viral or microbial antigen can be obtained, e.g., from the GenBank database or a database like it, literature publications, or by routine cloning and sequencing.

Examples of antibodies available useful for the diagnosis or treatment of viral infection or microbial infection include, but are not limited to, SYNAGIS (MedImmune, Inc., MD) which is a humanized anti-respiratory syncytial virus (RSV) monoclonal antibody useful for the treatment of patients with RSV infection; PRO542 (Progenics Pharmaceuticals, Inc., NY) which is a CD4 fusion antibody useful for the treatment of HIV infection; OSTAVIR (Protein Design Labs, Inc., CA) which is a human antibody useful for the treatment of hepatitis B virus; PROTOVIR (Protein Design Labs, Inc., CA) which is a humanized IgG1 antibody useful for the treatment of cytomegalovirus (CMV); and anti-LPS antibodies.

Other antibodies include, but are not limited to, antibodies against the antigens from pathogenic strains of bacteria (e.g., Streptococcus pyogenes, Streptococcus pneumoniae, Neisseria gonorrhea, Neisseria meningitidis, Corynebacterium diphtheriae, Clostridium botulinum, Clostridium perfringens, Clostridium tetani, Hemophilus influenzae, Klebsiella pneumoniae, Klebsiella ozaenas, Klebsiella rhinoscleromotis, Staphylococcaureus, Vibrio colerae, Escherichia coli, Pseudomonas aeruginosa, Campylobacter (Vibrio) fetus, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Pneumocystis carinii, Francisella tularensis, Brucella abortus, Brucella suis, Brucella melitensis, Mycoplasma spp., Rickettsia prowazeki, Rickettsia tsutsugumushi, Chlamydia spp.); pathogenic fungi (e.g., Coccidioides immitis, Aspergillus fumigatus, Candida albicans, Blastomyces dermatitidis, Cryptococcus neoformans, Histoplasma capsulatum); protozoa (Entomoeba histolytica, Toxoplasma gondii, Trichomonas tenas, Trichomonas hominis, Trichomonas vaginalis, Tryoanosoma gambiense, Trypanosoma rhodesiense, Trypanosoma cruzi, Leishmania donovani, Leishmania tropica, Leishmania braziliensis, Pneumocystis pneumonia, Plasmodium vivax, Plasmodium falciparum, Plasmodium malaria); or Helminiths (Enterobius vermicularis, Trichuris trichiura, Ascaris lumbricoides, Trichinella spiralis, Strongyloides stercoralis, Schistosoma japonicum, Schistosoma mansoni, Schistosoma haematobium, and hookworms).

Other antibodies include, but are not limited to, antibodies against antigens of pathogenic viruses, including as examples and not by limitation: Poxviridae, Herpesviridae, Herpes Simplex virus 1, Herpes Simplex virus 2, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Hepatitis A virus, Hepatitis B virus, Hepatitis C virus, Hepatitis E virus, Non A/Non B Hepatitis virus, Rhinoviridae, Coronaviridae, Rotoviridae, and Human Immunodeficiency Virus.

Methods for Introducing an Amino Acid Substitution into an Antibody by Altering the Nucleic Acid Sequence Encoding the Protein

An amino acid substitution can be introduced into a nucleic acid sequence encoding an antibody by any suitable method. Such methods include polymerase chain reaction-based mutagenesis, site-directed mutagenesis, gene synthesis using the polymerase chain reaction with synthetic DNA oligomers, and nucleic acid synthesis followed by ligation of the synthetic DNA into an expression vector, comprising other portions of the heavy and/or light chain, as applicable. (See also Sambrook et al. and Ausubel et al., supra)

A nucleotide sequence encoding an antibody can be obtained, for example, from the GenBank database or a similar database, literature publications, or by routine cloning and sequencing. Examples of some methods that can be used for directed mutagenesis are as follows: oligonucleotide directed mutagenesis with M13 DNA, oligonucleotide directed mutagenesis with plasmid DNA, and PCR-amplified oligonucleotide directed mutagenesis. (See, e.g., Glick et al., Molecular Biotechnology: Principles and Applications of Recombinant DNA, Second Edition, ASM Press, pp. 171-182 (1998). An example of mutagenesis and cloning is described in Example 1.

Detailed protocols for oligonucleotide-directed mutagenesis and related techniques for mutagenesis of cloned DNA are well-known (see, e.g., Zoller and Smith, Nucleic Acids Res. 10:6487-6500 (1982); see also Sambrook et al., supra; and Ausubel et al., supra).

In some embodiments, the amino acid substitution is a serine for a cysteine residue. In some embodiments, the amino acid substitution introduces is a serine or threonine residue. In some embodiments, the amino acid substitution introduces a neutral (e.g., serine, threonine or glycine) or hydrophilic (e.g., methionine, alanine, valine, leucine or isoleucine) residue. In some embodiments, the amino acid substitution introduces a natural amino acid, other than a cysteine residue.

Although the present invention provides methods for introducing an amino acid substitution of an interchain cysteine residue (e.g., a cysteine to serine substitution) into an antibody or antibody fragment, it will be understood that the present invention is not so limited. It will occur to those of ordinary skill in the art that it is possible to introduce/remove other amino acids for conjugation, such as lysine residues, at other positions of the antibody or antibody fragment. Also, a sulfhydryl group(s) can also be recombinantly introduced into an antibody at an amino acid other than an interchain cysteine residue. Suitable alternative mutagenesis sites for conjugation can be identified using molecular modeling techniques that are well-known to those of skill in the art. See, for example, Lesk et al., “Antibody Structure and Structural Predictions Useful in Guiding Antibody Engineering,” in Antibody Engineering: A Practical Guide, C. Borrebaeck (ed.), W.H. Freeman and Company, pp. 1-38 (1992); Cheetham, “Engineering Antibody Affinity,” Antibody Engineering: A Practical Guide (supra) at pp. 39-67. See generally Sambrook et al., Molecular Cloning, A Laboratory Manual, 3rd ed., Cold Spring Harbor Publish., Cold Spring Harbor, N.Y. (2001); Ausubel et al., Current Protocols in Molecular Biology, 4th ed., John Wiley and Sons, New York (1999) (all of which are incorporated by reference herein), for methods for site-directed mutagenesis.

### Methods for Expressing and Isolating the Protein Product of an Engineered Antibody DNA Sequence

A. Methods for Expressing an Engineered Antibody

After altering the nucleotide sequence, the nucleic acid is inserted into a cloning vector for further analysis, such as confirmation of the nucleic acid sequence. To express the polypeptide encoded by the nucleic acid, the nucleic acid can be operably linked to regulatory sequences controlling transcriptional expression in an expression vector, then introduced into a prokaryotic or eukaryotic host cell. In addition to transcriptional regulatory sequences, such as promoters and enhancers, expression vectors may include translational regulatory sequences and/or a marker gene which is suitable for selection of cells that contain the expression vector.

Promoters for expression in a prokaryotic host can be repressible, constitutive, or inducible. Suitable promoters are well-known to those of skill in the art and include, for example, promoters for T4, T3, Sp6 and T7 polymerases, the P_{R} and P_{L} promoters of bacteriophage lambda, the trp, recA, heat shock, and lacZ promoters of E. coli, the alpha-amylase and the sigma₂₈-specific promoters of B. subtilis, the promoters of the bacteriophages of Bacillus, Streptomyces promoters, the int promoter of bacteriophage lambda, the bla promoter of the beta-lactamase gene of pBR322, and the CAT promoter of the chloramphenicol acetyl transferase gene. Prokaryotic promoters are reviewed by Glick, J. Ind. Microbiol. 1:277-282 (1987); Watson et al., Molecular Biology Of The Gene, Fourth Edition, Benjamin Cummins (1987); Ausubel et al., supra; and Sambrook et al., supra.

In some embodiments, the prokaryotic host is E. coli. Suitable strains of E. coli include, for example, Y1088, Y1089, CSH18, ER1451 and ER1647 (see, e.g., Brown (Ed.), Molecular Biology Labfax, Academic Press (1991)). An alternative host is Bacillus subtilus, including such strains as BR151, YB886, MI119, MI120 and B170 (see, e.g., Hardy, “Bacillus Cloning Methods,” in DNA Cloning: A Practical Approach, Glover (Ed.), IRL Press (1985)).

Methods for producing antibody fragments in E. coli are well-known to those in the art. See, for example, Huse, “Combinatorial Antibody Expression Libraries in Filamentous Phage,” in Antibody Engineering: A Practical Guide, C. Borrebaeck (Ed.), W.H. Freeman and Company, pp. 103-120 (1992); Ward, “Expression and Purification of Antibody Fragments Using Escherichia coli as a Host,” id. at pp. 121-138 (1992). Fv fragments can also be produced by methods known in the art. See, e.g., id. See also Whitlow et al., “Single-Chain Fv Proteins and their Fusion Proteins,” in New Techniques In Antibody Generation, Methods 2(2) (1991). Moreover, certain expression systems for cloning antibodies in prokaryotic cells are commercially available.

In some embodiments, the nucleic acid sequence is expressed in eukaryotic cells, and especially mammalian, insect, and yeast cells. In one embodiment, the eukaryotic host is a mammalian cell. Mammalian cells provide post-translational modifications to the cloned polypeptide including proper folding and glycosylation. For example, such mammalian host cells include COS-7 cells (e.g., ATCC CRL 1651), non-secreting myeloma cells (e.g., SP2/0-AG14; ATCC CRL 1581), Chinese hamster ovary cells (e.g., CHO-K1, ATCC CCL 61; CHO-DG44, Urlaub et al., Somat Cell Mol. Genet. 12(6):555-66 (1986)), rat pituitary cells (e.g., GH₁; ATCC CCL 82), HeLa S3 cells (e.g., ATCC CCL 2.2), and rat hepatoma cells (e.g., H-4-II-E; ATCC CRL 1548).

For a mammalian host, the transcriptional and translational regulatory signals may be derived from viral sources, such as adenovirus, bovine papilloma virus, and simian virus. In addition, promoters from mammalian cells, such as actin, collagen, or myosin, can be employed. Alternatively, a prokaryotic promoter (such as the bacteriophage T3 RNA polymerase promoter) can be employed, wherein the prokaryotic promoter is regulated by a eukaryotic promoter (for example, see Zhou et al., Mol. Cell. Biol. 10:4529-4537 (1990); Kaufman et al., Nucl. Acids Res. 19:4485-4490 (1991)). Transcriptional initiation regulatory signals may be selected which allow for repression or activation, so that expression of the genes can be modulated.

In general, eukaryotic regulatory regions will include a promoter region sufficient to direct the initiation of RNA synthesis. Such a eukaryotic promoter can be, for example, the promoter of the mouse metallothionein I gene (Hamer et al., J. Mol. Appl. Gen. 1:273-288 (1982)); the TK promoter of Herpes virus (McKnight, Cell 31:355-365 (1982)); the SV40 early promoter (Benoist et al., Nature (London) 290:304-310 (1981)); the Rous sarcoma virus promoter ((Gorman, “High Efficiency Gene Transfer into Mammalian cells,” in DNA Cloning: A Practical Approach, Volume II, Glover (Ed.), IRL Press, pp. 143-190 (1985)); the cytomegalovirus promoter (Foecking et al., Gene 45:101 (1980)); the yeast gal4 gene promoter (Johnston et al., Proc. Natl. Acad. Sci. USA 79:6971-6975 (1982); Silver et al., Proc. Natl. Acad. Sci. USA 81:5951-5955 (1984)); and the IgG promoter (Orlandi et al., Proc. Natl. Acad. Sci. USA 86:3833-3837 (1989)).

Strong regulatory sequences can be used. Examples of such regulatory sequences include the SV40 promoter-enhancer (Gorman, “High Efficiency Gene Transfer into Mammalian cells,” in DNA Cloning: A Practical Approach, Volume II, Glover (Ed.), IRL Press, pp. 143-190 (1985)), the hCMV-MIE promoter-enhancer (Bebbington et al., Bio/Technology 10:169-175 (1992)), Chinese Hamster EF-1α promoter (see, e.g., U.S. Pat. No. 5,888,809) and antibody heavy chain promoter (Orlandi et al., Proc. Natl. Acad. Sci. USA 86:3833-3837 (1989)). Also included are the kappa chain enhancer for the expression of the light chain and the IgH enhancer (Gillies, “Design of Expression Vectors and Mammalian Cell Systems Suitable for Engineered Antibodies,” in Antibody Engineering: A Practical Guide, C. Borrebaeck (Ed.), W.H. Freeman and Company, pp. 139-157 (1992); Orlandi et al., supra).

The engineered antibody-encoding nucleic acid and an operably linked promoter may be introduced into eukaryotic cells as a non-replicating DNA molecule, which may either be a linear molecule or a circular molecule. Since such molecules are incapable of autonomous replication, the expression of the protein may occur through the transient expression of the introduced sequence. In one aspect, permanent expression occurs through the integration of the introduced sequence into the host chromosome.

In some embodiments, the introduced nucleic acid will be incorporated into a plasmid or viral vector that is capable of autonomous replication in the recipient host. Numerous possible vector systems are available for this purpose. One class of vectors utilize DNA elements which provide autonomously replicating extra-chromosomal plasmids, derived from animal viruses such as bovine papilloma virus, polyoma virus, adenovirus, or SV40 virus. A second class of vectors relies upon the integration of the desired genomic or cDNA sequences into the host chromosome. Additional elements may also be needed for optimal synthesis of mRNA. These elements may include splice signals, as well as transcription promoters, enhancers, and termination signals. The cDNA expression vectors incorporating such elements include those described by Okayama, Mol. Cell. Biol. 3:280 (1983), Sambrook et al., supra, Ausubel et al., supra, Bebbington et al., supra, Orlandi et al., supra, Fouser et al., Bio/Technology 10:1121-1127 (1992); and Gillies, supra. Genomic DNA expression vectors which include intron sequences are described by Orlandi et al., supra. Also, see generally, Lerner et al. (Eds.), New Techniques In Antibody Generation, Methods 2(2) (1991).

To obtain mammalian cells that express intact antibody, the expression vector comprising a nucleic acid encoding an antibody light chain can be co-transfected or transfected into mammalian cells with an antibody heavy chain expression vector.

Alternatively, mammalian cells containing a heavy chain expression vector can be transfected with an antibody light chain expression vector, or mammalian cells containing an antibody light chain expression vector can be transfected with an antibody heavy chain expression vector. Moreover, mammalian cells can be transfected with a single expression vector comprising nucleic acid (e.g., DNA) fragments that encode an antibody light chain, as well as nucleic acid (e.g., DNA) fragments that encode antibody heavy chain. See, for example, Gillies, supra; Bebbington et al., supra. Any of these approaches will produce transfected cells that express whole engineered antibody molecules. Standard transfection and transformation techniques are well known in the art. See, for example, Sambrook et al., supra; Ausubel et al., supra.

An example of cell line development and protein expression is described in Example 1.

B. Methods for Isolating an Engineered Antibody from Transfected Cells

Transformed or transfected cells that carry the expression vector are selected using the appropriate drug. For example, G418 can be used to select transfected cells carrying an expression vector having the aminoglycoside phosphotransferase gene. (See, e.g., Southern et al., J. Mol. Appl. Gen. 1:327-341 (1982).) Alternatively, hygromycin-B can be used to select transfected cells carrying an expression vector having the hygromycin-B-phosphotransferase gene. (See, e.g., Palmer et al., Proc. Natl. Acad. Sci. USA 84:1055-1059 (1987).) Aminopterin and mycophenolic acid can be used to select transfected cells carrying an expression vector having the xanthine-guanine phosphoribosyltransferase gene. (See, e.g., Mulligan et al., Proc. Natl. Acad. Sci. USA 78:2072-2076 (1981).) Methotrexate can be used to select transformed cells carrying an expression vector having the dihydrofolate reductase gene. (See, e.g., Wigler et al., Proc Natl. Acad. Sci. USA 77(6):3567-70 (1980).)

Transformed or transfected cells that produce the engineered antibody can be identified using a variety of methods. For example, any immunodetection assay can be used to identify such “transfectomas.”

After transformants or transfectants have been identified, the cells are cultured and antibodies are isolated from the cells and/or the culture supernatants. Isolation techniques include affinity chromatography with Protein-A Sepharose, size-exclusion chromatography, and ion-exchange chromatography. For example, see Coligan et al. (eds.), Current Protocols In Immunology, John Wiley and Sons (1991), for detailed protocols.

### Methods for Preparing Immunoconjugates

A. Preparation of Antibody Fragments

The present invention also provides immunoconjugates of engineered antibodies or from antigen-binding antibody fragments. Antibody fragments can be obtained from, for example, recombinant host cells (e.g., transformants or transfectants) and/or by proteolytic cleavage of intact engineered antibodies. Antibody fragments can be obtained directly from transformants or transfectants by transfecting cells with a heavy chain structural gene that has been mutated. For example, transfectomas can produce Fab fragments if a stop codon is inserted following the sequence of the C_{H}1 domain. Alternatively, transfectomas can produce Fab′ or F(ab′)₂ fragments if a stop codon is inserted after the sequence encoding the hinge region of the heavy chain.

Alternatively, antibody fragments can be prepared from intact antibodies using well-known proteolytic techniques. For example, see, Coligan et al., supra. Moreover, F(ab′)₂ fragments can be obtained using pepsin digestion of intact antibodies. Divalent fragments can be cleaved to monovalent fragments using conventional disulfide bond reducing agents, e.g., dithiothreitol (DTT) and the like.

B. Methods of Conjugation

A wide variety of diagnostic, preventative and therapeutic agents can be advantageously conjugated to the antibodies of the invention. In some embodiments, can antibody can be stoichiometrically or fully-loaded (i.e., C#=Y, where Y refers to the average number of active moieties attached to each antibody molecule). In other embodiments, an antibody can be partially-loaded (i.e., C#>Y).

Immunoconjugates can be prepared by conjugating a diagnostic, preventative or therapeutic agent to an intact antibody, or antigen-binding fragment thereof. Such techniques are described in Shih et al., Int. J. Cancer 41:832-839 (1988); Shih et al., Int. J. Cancer 46:1101-1106 (1990); Shih et al., U.S. Pat. No. 5,057,313; Shih Cancer Res. 51:4192, International Publication WO 02/088172; U.S. Pat. No. 6,884,869; International Patent Publication WO 2005/081711; and U.S. Published Application 2003-0130189 A1, all of which are incorporated by reference herein.

In addition, those of skill in the art will recognize numerous possible variations of conjugation methods. For example, it is possible to construct a “divalent immunoconjugate” by attaching a diagnostic or therapeutic agent to a carbohydrate moiety and to a free sulfhydryl group.

In some embodiments, the interchain cysteine residues are present as a disulfide bond as a result of the oxidation of the thiol (—SH) side groups of the cysteine residues.

Treatment of the disulfide bond with a reducing agent can causes reductive cleavage of the disulfide bonds to leave free thiol groups.

In some embodiments, the agent has, or is modified to include, a group reactive with an interchain cysteine residue. For example, an agent can be attached by conjugation to thiols. For examples of chemistries that can be used for conjugation, see, e.g., Current Protocols in Protein Science (John Wiley & Sons, Inc.), Chapter 15 (Chemical Modifications of Proteins) (the disclosure of which is incorporated by reference herein in its entirety).

For example, when chemical activation of the antibody results in formation of free thiol groups, the protein may be conjugated with a sulfhydryl reactive agent. In some embodiments, the agent is one which is substantially specific for free thiol groups. Such agents include, for example, malemide, haloacetamides (e.g., iodo, bromo or chloro), haloesters (e.g., iodo, bromo or chloro), halomethyl ketones (e.g., iodo, bromo or chloro), benzylic halides (e.g., iodide, bromide or chloride), vinyl sulfone and pyridylthio.

In specific embodiments, the sulfhydryl reactive agent can be an alpha-haloacetyl compounds such as iodoacetamide, maleimides such as N-ethylmaleimide, mercury derivatives such as 3,6-bis-(mercurimethyl)dioxane with counter ions of acetate, chloride or nitrate, and disulfide derivatives such as disulfide dioxide derivatives, polymethylene bismethane thiosulfonate reagents and crabescein (a fluorescent derivative of fluorescein containing two free sulfhydryl groups which have been shown to add across disulfide bonds of reduced antibody).

Alpha-haloacetyl compounds such as iodoacetate readily react with sulfhydryl groups to form amides. These compounds have been used to carboxymethylate free thiols. They are not strictly SH specific and will react with amines. The reaction involves nucleophilic attack of the thiolate ion resulting in a displacement of the halide. The reactive haloacetyl moiety, X—CH₂CO—, has been incorporated into compounds for various purposes. For example, bromotrifluoroacetone has been used for F-19 incorporation, and N-chloroacetyliodotyramine has been employed for the introduction of radioactive iodine into proteins.

Maleimides such as N-ethylmaleimide are considered to be fairly specific to sulfhydryl groups, especially at pH values below 7, where other groups are protonated. Thiols undergo Michael reactions with maleimides to yield exclusively the adduct to the double bond. The resulting thioether bond is very stable. They also react at a much slower rate with amino and imidazoyl groups. At pH 7, for example, the reaction with simple thiols is about 1,000 fold faster than with the corresponding amines. The characteristic absorbance change in the 300 nm region associated with the reaction provides a convenient method for monitoring the reaction. These compounds are stable at low pH but are susceptible to hydrolysis at high pH. See generally Wong, Chemistry of Protein Conjugation and Cross-linking; CRC Press, Inc., Boca Raton, 1991: Chapters 2 and 4.

An agent (such as a drug) which is not inherently reactive with sulfhydryls may still be conjugated to the chemically activated antibody by means of a bifunctional crosslinking agent which bears both a group reactive with the agent and a sulfhydryl reactive group. The cross-linking agent may be reacted simultaneously with both the molecule of interest (e.g., through an amino, carboxy or hydroxy group) and the chemically activated protein, or it may be used to derivatize the molecule of interest to form a partner molecule which is then sulfhydryl reactive by virtue of a moiety derived from the agent, or it may be used to derivatize the chemically activated protein to make it reactive with the molecule of interest.

The agent also can be linked to an antibody by a linker. Suitable linkers include, for example, cleavable and non-cleavable linkers. A cleavable linker is typically susceptible to cleavage under intracellular conditions. Suitable cleavable linkers include, for example, a peptide linker cleavable by an intracellular protease, such as lysosomal protease or an endosomal protease. In exemplary embodiments, the linker can be a dipeptide linker, such as a valine-citrulline (val-cit) or a phenylalanine-lysine (phe-lys) linker. Other suitable linkers include linkers hydrolyzable at a pH of less than 5.5, such as a hydrazone linker. Additional suitable cleavable linkers include disulfide linkers.

A linker can include a group for linkage to the antibody. For example, a linker can include a sulfhydryl reactive group(s) (e.g., malemide, haloacetamides (e.g., iodo, bromo or chloro), haloesters (e.g., iodo, bromo or chloro), halomethyl ketones (e.g., iodo, bromo or chloro), benzylic halides (e.g., iodide, bromide or chloride), vinyl sulfone and pyridylthio). See generally Wong, Chemistry of Protein Conjugation and Cross-linking; CRC Press, Inc., Boca Raton, 1991.

In certain embodiments, the immunoconjugate has the following formula:

Ab_{z}Aₐ-W_{w}-Y_{y}-D)ₚ
or pharmaceutically acceptable salts or solvates thereof,
wherein:
Ab is an antibody,
A is a stretcher unit,
a is 0 or 1,
each W is independently a linker unit,
w is an integer ranging from 0 to 12,
Y is a spacer unit, and
y is 0, 1 or 2,
p ranges from 1 to about 20, and
D is a diagnostic, preventative and therapeutic agent,
z is the number of predetermined conjugation sites on the protein.

When the antibody is fully loaded, p=z. When the antibody is partially loaded, p<z. In some embodiments, p is an even integer. In specific embodiments, p=2, 4, 6 or 8. In a specific embodiment, p=z=4. In other embodiments, 0<p<8.

A stretcher unit can is capable of linking a linker unit to an antibody. The stretcher unit has a functional group that can form a bond with an interchain cysteine residue of the antibody. Useful functional groups include, but are not limited to, sulfhydryl reactive groups, as described above.

The linker unit is typically an amino acid unit, such as for example a dipeptide, tripeptide, tetrapeptide, pentapeptide, hexapeptide, heptapeptide, octapeptide, nonapeptide, decapeptide, undecapeptide or dodecapeptide unit. The linker unit can be cleavage or non-cleavable inside the cell.

In one embodiment, the amino acid unit is valine-citrulline. In another embodiment, the amino acid unit is phenylalanine-lysine. In yet another embodiment, the amino acid unit is N-methylvaline-citrulline. In yet another embodiment, the amino acid unit is 5-aminovaleric acid, homo phenylalanine lysine, tetraisoquinolinecarboxylate lysine, cyclohexylalanine lysine, isonepecotic acid lysine, beta-alanine lysine, glycine serine valine glutamine and isonepecotic acid. In certain embodiments, the Amino Acid unit can comprise natural amino acids. In other embodiments, the Amino Acid unit can comprise non-natural amino acids.

A spacer unit, if present, links a linker unit to D. Alternately, a spacer unit can link a stretcher unit to a drug moiety when the linker unit is absent. The spacer unit can also link a diagnostic, preventative and therapeutic agents to an antibody when both the linker unit and stretcher unit are absent. In one embodiment, the spacer unit is a p-aminobenzyl alcohol (PAB) unit, a p-aminobenzyl ether unit, or p-aminobenzyl carbamoyl unit. (See, e.g., U.S. Patent Publication Nos. 2003-0130189).

In some embodiments, the immunoconjugate has the formula:

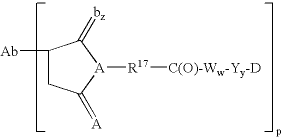

wherein R¹⁷ is selected from —C₁-C₁₀ alkylene-, —C₃-C₈ carbocyclo-, —O—(C₁-C₈ alkyl)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —(CH₂CH₂O)ᵣ—, and —(CH₂CH₂O)ᵣ—CH₂—. In some embodiments, R¹⁷ is —(CH₂)₅— or (CH₂CH₂O)ᵣ—CH₂— and r is 2.

In another embodiment, the immunoconjugate has the formula:

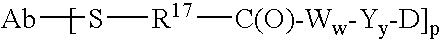

wherein R¹⁷ is as defined above.

In additional embodiments, the immunoconjugate has one of the following formulae:

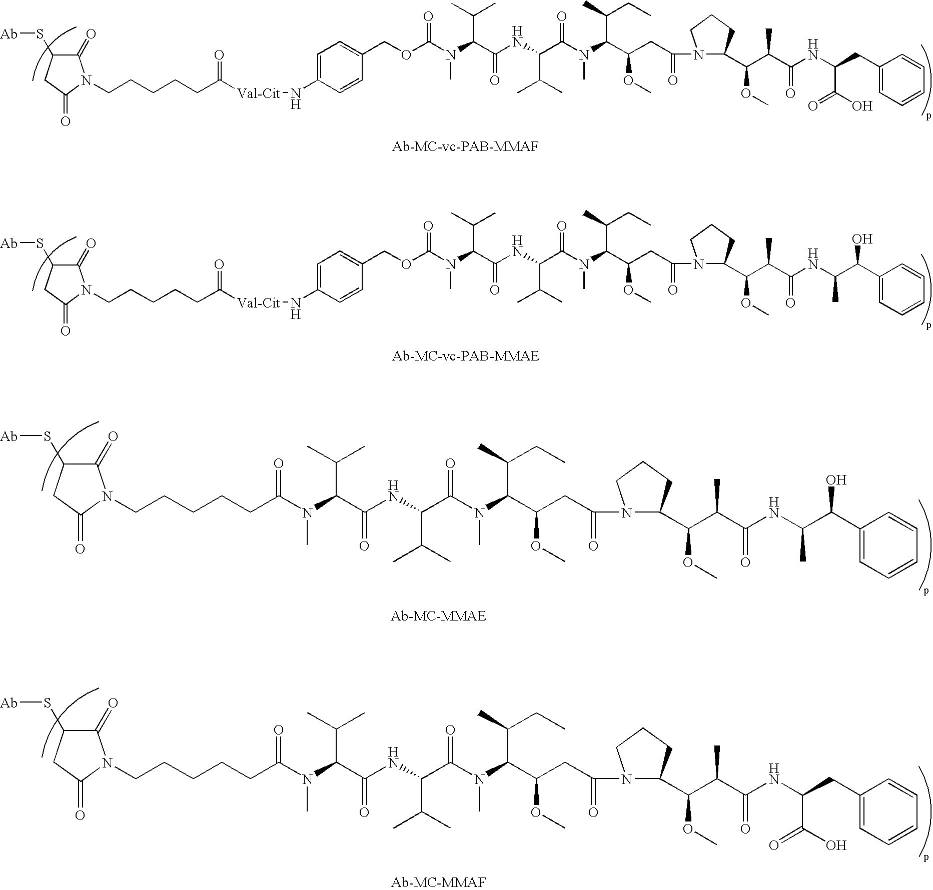

The final immunoconjugate may be purified using conventional techniques, such as sizing chromatography on Sephacryl S-300, affinity chromatography such as protein A or protein G sepharose, or the like.

Examples of protein purification and conjugation is described in Examples 1 and 2.

### Use of Immunoconjugates for Diagnosis and Therapy

A. Use of Immunoconjugates for Diagnosis

The immunoconjugates can be used for diagnostic imaging. For example, the immunoconjugate can be a radiolabeled monoclonal antibody. See, for example, Srivastava (ed.), Radiolabeled Monoclonal Antibodies For Imaging And Therapy, Plenum Press (1988); Chase, “Medical Applications of Radioisotopes,” in Remington's Pharmaceutical Sciences, 18th Edition, Gennaro et al. (eds.), Mack Publishing Co., pp. 624-652 (1990); and Brown, “Clinical Use of Monoclonal Antibodies,” in Biotechnology and Pharmacy, Pezzuto et al. (eds.), Chapman and Hall, pp. 227-249 (1993). This technique, also known as immunoscintigraphy, uses a gamma camera to detect the location of gamma-emitting radioisotopes conjugated to monoclonal antibodies. Diagnostic imaging can be used to diagnose cancer, autoimmune disease, infectious disease and/or cardiovascular disease. (See, e.g., Brown, supra.)

In one example, the immunoconjugates can be used to diagnose cardiovascular disease. For example, immunoconjugates comprising anti-myosin antibody fragments can be used for imaging myocardial necrosis associated with acute myocardial infarction. Immunoconjugates comprising antibody fragments that bind to platelets or fibrin can be used for imaging deep-vein thrombosis. Moreover, immunoconjugates comprising antibody fragments that bind to activated platelets can be used for imaging atherosclerotic plaque.

Immunoconjugates can also be used in the diagnosis of infectious diseases. For example, immunoconjugates comprising antibody fragments that bind specific bacterial antigens can be used to localize abscesses. In addition, immunoconjugates comprising antibody fragments that bind granulocytes and inflammatory leukocytes can be used to localize sites of bacterial infection.

Numerous studies have evaluated the use of monoclonal antibodies for scintigraphic detection of cancer. See, for example, Brown, supra. Investigations have covered the major types of solid tumors such as melanoma, colorectal carcinoma, ovarian carcinoma, breast carcinoma, sarcoma, and lung carcinoma. Thus, the present invention also contemplates the detection of cancer using immunoconjugates comprising antibody fragments that bind tumor markers to detect cancer. Examples of such tumor markers include carcinoembryonic antigen, alpha-fetoprotein, oncogene products, tumor-associated cell surface antigens, and necrosis-associated intracellular antigens, as well as the tumor-associated antigens and tumor-specific antigens discussed infra.

In addition to diagnosis, monoclonal antibody imaging can be used to monitor therapeutic responses, detect recurrences of a disease, and guide subsequent clinical decisions.

For diagnostic and monitoring purposes, radioisotopes may be bound to antibody fragments either directly or indirectly by using an intermediary functional group. Such intermediary functional groups include, for example, DTPA (diethylenetriaminepentaacetic acid) and EDTA (ethylene diamine tetraacetic acid). The radiation dose delivered to the patient is typically maintained at as low a level as possible. This may be accomplished through the choice of isotope for the best combination of minimum half-life, minimum retention in the body, and minimum quantity of isotope which will permit detection and accurate measurement. Examples of radioisotopes which can be bound to antibodies and are appropriate for diagnostic imaging include ⁹⁹mTc and ¹¹¹In.

Studies indicate that antibody fragments, particularly Fab and Fab′, provide suitable tumor/background ratios. (See, e.g., Brown, supra.)

The immunoconjugates also can be labeled with paramagnetic ions for purposes of in vivo diagnosis. Elements which are particularly useful for Magnetic Resonance Imaging include Gd, Mn, Dy, and Fe ions.

The immunoconjugates can also detect the presence of particular antigens in vitro. In such immunoassays, the immunoconjugates may be utilized in liquid phase or bound to a solid-phase carrier. For example, an intact antibody, or antigen-binding fragment thereof, can be attached to a polymer, such as aminodextran, in order to link the antibody component to an insoluble support such as a polymer-coated bead, plate, or tube.

Alternatively, the immunoconjugates can be used to detect the presence of particular antigens in tissue sections prepared from a histological specimen. Such in situ detection can be accomplished, for example, by applying a detectably-labeled immunoconjugate to the tissue sections. In situ detection can be used to determine the presence of a particular antigen and to determine the distribution of the antigen in the examined tissue. General techniques of in situ detection are well known to those of ordinary skill. (See, e.g., Ponder, “Cell Marking Techniques and Their Application,” in Mammalian Development: A Practical Approach, Monk (ed.), IRL Press, pp. 115-138 (1987); Coligan et al., supra.)

Detectable labels such as enzymes, fluorescent compounds, electron transfer agents, and the like can be linked to a carrier by conventional methods well known to the art. These labeled carriers and the immunoconjugates prepared from them can be used for in vitro immunoassays and for in situ detection, much as an antibody conjugate can be prepared by direct attachment of the labels to antibody. The loading of the immunoconjugate with a plurality of labels can increase the sensitivity of immunoassays or histological procedures, where only a low extent of binding of the antibody, or antibody fragment, to target antigen is achieved.

B. Use of Immunoconjugates for Therapy

Immunoconjugates can be used to treat viral and bacterial infectious diseases, cardiovascular disease, autoimmune disease, and cancer. The objective of such therapy is to deliver cytotoxic or cytostatic doses of an active agent (e.g., radioactivity, a toxin, or a drug) to target cells, while minimizing exposure to non-target tissues.

A radioisotope can be attached to an intact antibody, or antigen-binding fragment thereof, directly or indirectly, via a chelating agent. For example, ⁶⁷Cu can be conjugated to an antibody component using the chelating agent, p-bromo-acetamidobenzyl-tetraethylaminetetraacetic acid (TETA). (See, e.g., Chase, supra.)

Moreover, immunoconjugates can be prepared in which the therapeutic agent is a toxin or drug. Useful toxins for the preparation of such immunoconjugates include ricin, abrin, pokeweed antiviral protein, gelonin, diphtherin toxin, and Pseudomonas endotoxin. Useful chemotherapeutic drugs for the preparation of immunoconjugates include auristatin, dolastatin, MMAE, MMAF, AFP, AEB, doxorubicin, daunorubicin, methotrexate, melphalan, chlorambucil, vinca alkaloids, 5-fluorouridine, mitomycin-C, taxol, L-asparaginase, mercaptopurine, thioguanine, hydroxyurea, cytarabine, cyclophosphamide, ifosfamide, nitrosoureas, cisplatin, carboplatin, mitomycin, dacarbazine, procarbazine, topotecan, nitrogen mustards, cytoxan, etoposide, BCNU, irinotecan, camptothecins, bleomycin, idarubicin, dactinomycin, plicamycin, mitoxantrone, asparaginase, vinblastine, vincristine, vinorelbine, paclitaxel, and docetaxel and salts, solvents and derivatives thereof. Other suitable agents include chelators, such as DTPA, to which detectable labels such as fluorescent molecules or cytotoxic agents such as heavy metals or radionuclides can be complexed; and toxins such as Pseudomonas exotoxin, and the like.

In some embodiments, the diagnostic, preventative or therapeutic agent is auristatin E (also known in the art as dolastatin-10) or a derivative thereof as well as pharmaceutically salts or solvates thereof. Typically, the auristatin E derivative is, e.g., an ester formed between auristatin E and a keto acid. For example, auristatin E can be reacted with paraacetyl benzoic acid or benzoylvaleric acid to produce AEB and AEVB, respectively. Other typical auristatin derivatives include AFP, MMAF, and MMAE. The synthesis and structure of auristatin E and its derivatives, as well as linkers, are described in U.S. patent application Ser. No. 09/845,786 (U.S. Patent Application Publication No. 20030083263), U.S. Patent Application Publication No. 2005-0238629; International Patent Application No. PCT/US03/24209; International Patent Application No. PCT/US02/13435; International Patent Application No. PCT/US02/13435; International Patent Publication No. WO 04/073656; and U.S. Pat. Nos. 6,884,869; 6,323,315; 6,239,104; 6,214,345; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414 (all of which are incorporated by reference herein in their entirety).

In some embodiments, the anti-cancer agent includes, but is not limited to, a drug listed in Drug Table below.

Drug Table

| | |
|---|---|
| Alkylating agents | |
| Nitrogen mustards: | cyclophosphamide |
| | Ifosfamide |
| | trofosfamide |
| | Chlorambucil |
| Nitrosoureas: | carmustine (BCNU) |
| | Lomustine (CCNU) |
| Alkylsulphonates | busulfan |
| | Treosulfan |
| Triazenes: | Dacarbazine |
| Platinum containing compounds: | Cisplatin |
| | carboplatin |
| Plant Alkaloids | |
| Vinca alkaloids: | vincristine |
| | Vinblastine |
| | Vindesine |
| | Vinorelbine |
| Taxoids: | paclitaxel |
| | Docetaxol |
| DNA Topoisomerase Inhibitors | |
| Epipodophyllins: | etoposide |
| | Teniposide |
| | Topotecan |
| | 9-aminocamptothecin |
| | camptothecin |
| | crisnatol |
| mitomycins: | Mitomycin C |
| Anti-metabolites | |
| Anti-folates: | |
| DHFR inhibitors: | methotrexate |
| | Trimetrexate |
| IMP dehydrogenase Inhibitors: | mycophenolic acid |
| | Tiazofurin |
| | Ribavirin |
| | EICAR |
| Ribonuclotide reductase Inhibitors: | hydroxyurea |
| | deferoxamine |
| Pyrimidine analogs: | |
| Uracil analogs | 5-Fluorouracil |
| | Floxuridine |
| | Doxifluridine |
| | Ratitrexed |
| Cytosine analogs | cytarabine (ara C) |
| | Cytosine arabinoside |
| | fludarabine |
| Purine analogs: | mercaptopurine |
| | Thioguanine |
| Hormonal therapies: | |
| Receptor antagonists: | |
| Anti-estrogen | Tamoxifen |
| | Raloxifene |
| | megestrol |
| LHRH agonists: | goscrclin |
| | Leuprolide acetate |
| Anti-androgens: | flutamide |
| | bicalutamide |
| Retinoids/Deltoids | |
| Vitamin D3 analogs: | EB 1089 |
| | CB 1093 |
| | KH 1060 |
| Photodynamic therapies: | vertoporfin (BPD-MA) |
| | Phthalocyanine |
| | photosensitizer Pc4 |
| | Demethoxy-hypocrellin A |
| | (2BA-2-DMHA) |
| Cytokines: | Interferon-α |
| | Interferon-γ |
| | Tumor necrosis factor |
| Others: | |
| Isoprenylation inhibitors: | Lovastatin |
| Dopaminergic neurotoxins: | 1-methyl-4-phenylpyridinium ion |
| Cell cycle inhibitors: | staurosporine |
| Actinomycins: | Actinomycin D |
| | Dactinomycin |
| Bleomycins: | bleomycin A2 |
| | Bleomycin B2 |
| | Peplomycin |
| Anthracyclines: | daunorubicin |
| | Doxorubicin (adriamycin) |
| | Idarubicin |
| | Epirubicin |
| | Pirarubicin |
| | Zorubicin |
| | Mitoxantrone |
| MDR inhibitors: | verapamil |
| Ca²⁺ ATPase inhibitors: | thapsigargin |

In some embodiments, the diagnostic, preventative or therapeutic agent is not a radioisotope.

In some embodiments, an immunoconjugate can be used to treat one of the following particular types of cancer:
Solid tumors, including but not limited to:
sarcoma
fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon cancer
colorectal cancer
kidney cancer
pancreatic cancer
bone cancer
breast cancer
ovarian cancer
prostate cancer
esophageal cancer
stomach cancer (e.g., gastrointestinal cancer)
oral cancer
nasal cancer
throat cancer
squamous cell carcinoma (e.g., of the lung)
basal cell carcinoma
adenocarcinoma (e.g., of the lung)
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
uterine cancer
testicular cancer
small cell lung carcinoma
bladder carcinoma
lung cancer
non-small cell lung cancer
epithelial carcinoma
glioma
glioblastoma multiforme
astrocytoma
medulloblastoma
craniopharyngioma
ependymoma
pinealoma
hemangioblastoma
acoustic neuroma
oligodendroglioma
meningioma
skin cancer
melanoma
neuroblastoma
retinoblastoma
blood-borne cancers, including but not limited to:
acute lymphoblastic leukemia “ALL”
acute lymphoblastic B-cell leukemia
acute lymphoblastic T-cell leukemia
acute myeloblastic leukemia “AML”
acute promyelocytic leukemia “APL”
acute monoblastic leukemia
acute erythroleukemic leukemia
acute megakaryoblastic leukemia
acute myelomonocytic leukemia
acute nonlymphocytic leukemia
acute undifferentiated leukemia
chronic myelocytic leukemia “CML”
chronic lymphocytic leukemia “CLL”
hairy cell leukemia
multiple myeloma
acute and chronic leukemias:
lymphoblastic
myelogenous
lymphocytic myelocytic leukemias
Lymphomas:
Hodgkin's disease
non-Hodgkin's Lymphoma
Multiple myeloma
Waldenström's macroglobulinemia
Heavy chain disease
Polycythemia vera
Other cancers:
Peritoneal cancer
Hepatocellular cancer
Hepatoma
Salivary cancer
Vulval cancer
Thyroid
Penile cancer
Anal cancer
Head and neck cancer
Renal cell carcinoma
Acute anaplastic large cell carcinoma
Cutaneous anaplastic large cell carcinoma

In some embodiments, an immunoconjugate can be used to treat one of the following particular types of autoimmune disease:
Active Chronic Hepatitis
Addison's Disease
Allergic Alveolitis
Allergic Reaction
Allergic Rhinitis
Alport's Syndrome
Anaphylaxis
Ankylosing Spondylitis
Anti-phospholipid Syndrome
Arthritis
Ascariasis
Aspergillosis
Atrophic Allergy
Atrophic Dermatitis
Atrophic Rhinitis
Behcet's Disease
Bird-Fancier's Lung
Bronchial Asthma
Caplan's Syndrome
Cardiomyopathy
Celiac Disease
Chagas' Disease
Chronic Glomerulonephritis
Cogan's Syndrome
Cold Agglutinin Disease
Congenital Rubella Infection
CREST Syndrome
Crohn's Disease
Cryoglobulinemia
Cushing's Syndrome
Dermatomyositis
Discoid Lupus
Dressler's Syndrome
Eaton-Lambert Syndrome
Echovirus Infection
Encephalomyelitis
Endocrine ophthalmopathy
Epstein-Barr Virus Infection
Equine Heaves
Erythematosis
Evan's Syndrome
Felty's Syndrome
Fibromyalgia
Fuch's Cyclitis
Gastric Atrophy
Gastrointestinal Allergy
Giant Cell Arteritis
Glomerulonephritis
Goodpasture's Syndrome
Graft v. Host Disease
Graves' Disease
Guillain-Barre Disease
Hashimoto's Thyroiditis
Hemolytic Anemia
Henoch-Schonlein Purpura
Idiopathic Adrenal Atrophy
Idiopathic Pulmonary Fibritis
IgA Nephropathy
Inflammatory Bowel Diseases
Insulin-dependent Diabetes Mellitus
Juvenile Arthritis
Juvenile Diabetes Mellitus (Type I)
Lambert-Eaton Syndrome
Laminitis
Lichen Planus
Lupoid Hepatitis
Lupus
Lymphopenia
Meniere's Disease
Mixed Connective Tissue Disease
Multiple Sclerosis
Myasthenia Gravis
Pernicious Anemia
Polyglandular Syndromes
Presenile Dementia
Primary Agammaglobulinemia
Primary Biliary Cirrhosis
Psoriasis
Psoriatic Arthritis
Raynauds Phenomenon
Recurrent Abortion
Reiter's Syndrome
Rheumatic Fever
Rheumatoid Arthritis
Sampter's Syndrome
Schistosomiasis
Schmidt's Syndrome
Scleroderna
Shulman's Syndrome
Sjorgen's Syndrome
Stiff-Man Syndrome
Sympathetic Ophthalmia
Systemic Lupus Erythematosis
Takayasu's Arteritis
Temporal Arteritis
Thyroiditis
Thrombocytopenia
Thyrotoxicosis
Toxic Epidermal Necrolysis
Type B Insulin Resistance
Type I Diabetes Mellitus
Ulcerative Colitis
Uveitis
Vitiligo
Waldenstrom's Macroglobulemia
Wegener's Granulomatosis

The use of the immunoconjugates for the treatment of other cancers or autoimmune disorders is also contemplated and within the scope of the present invention.

C. Administration of Immunoconjugates

Generally, the dosage of administered immunoconjugate will vary depending upon such factors as the patient's age, weight, height, sex, general medical condition, and previous medical history. Typically, it is desirable to provide the recipient with a dosage of immunoconjugate which is in the range of from about 1 pg/kg to 20 mg/kg (amount of agent/body weight of patient), although a lower or higher dosage may also be administered. For example, many studies have demonstrated successful diagnostic imaging with doses of 0.1 to 1.0 milligram, while other studies have shown improved localization with doses in excess of 10 milligrams. (See, e.g., Brown, supra.)

For therapeutic applications, generally about 10-200 milligrams of immunoconjugate will be administered, depending on protocol. In some embodiments, a dose is from about 0.5 mg/kg to about 20 mg/kg, or about 1 mg/kg to about 10 mg/kg or about 15 mg/kg. Some protocols provide for the administration daily for a period of several days, several weeks or several months. In some embodiments, an immunoconjugate is administered daily, 1-3 times per week, weekly, biweekly or monthly. To reduce patient sensitivity, it may be necessary to reduce the dosage and/or use antibodies from other species and/or use hypoallergenic antibodies, e.g., hybrid human or primate antibodies.

Administration of immunoconjugates to a patient can be intravenous, intraarterial, intraperitoneal, intramuscular, subcutaneous, intrapleural, intrathecal, by perfusion through a regional catheter, or by direct intralesional injection. When administering immunoconjugates by injection, the administration may be by continuous infusion, or by single or multiple boluses.

The immunoconjugates can be formulated according to known methods to prepare pharmaceutically useful compositions, such as a medicament, whereby immunoconjugates are combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. (See, e.g., Remington's Pharmaceutical Sciences, 18th Ed. (1990).)

For purposes of immunotherapy, an immunoconjugate and a pharmaceutically acceptable carrier are administered to a patient in a therapeutically effective amount. A “therapeutically effective amount” is the amount administered that is physiologically significant. An agent is physiologically significant if its presence results in a detectable change in the physiology of a recipient patient.

Additional pharmaceutical methods may be employed to control the duration of action of an immunoconjugate in a therapeutic application. Control release preparations can be prepared through the use of polymers to complex or adsorb an immunoconjugate. For example, biocompatible polymers include matrices of poly(ethylene-co-vinyl acetate) and matrices of a polyanhydride copolymer of a stearic acid dimer and sebacic acid. (See, e.g., Sherwood et al., Bio/Technology 10:1446-1449 (1992).) The rate of release of an immunoconjugate from such a matrix depends upon the molecular weight of the immunoconjugate, the amount of immunoconjugate within the matrix, and the size of dispersed particles. (See, e.g., Saltzman et al., Biophysical. J. 55:163-171 (1989); and Sherwood et al., supra.) Other solid dosage forms are described in Remington's Pharmaceutical Sciences, 18th Ed. (1990).

The present invention is not to be limited in scope by the specific embodiments described herein. Various modifications of the invention in addition to those described herein will become apparent to those skilled in the art from the foregoing description and accompanying figures. Such modifications are intended to fall within the scope of the appended claims.

The invention is further described in the following examples, which are not intended to limit the scope of the invention. Cell lines described in the following examples were maintained in culture according to the conditions specified by the American Type Culture Collection (ATCC) or Deutsche Sammlung von Mikroorganismen und Zellkulturen GmbH, Braunschweig, Germany (DMSZ). Cell culture reagents were obtained from Invitrogen Corp., Carlsbad, Calif.

### EXAMPLES

### Example 1

### Construction and Expression of CAC10 Cysteine Variants

### Procedures

Construction of chimeric AC10 (cAC10) from the AC10 hybridoma and expression of cAC10 in a CHO cell line has been described (Wahl et al., Cancer Res. 62(13): 3736-42 (2002)).

(i) Mutagenesis and Cloning

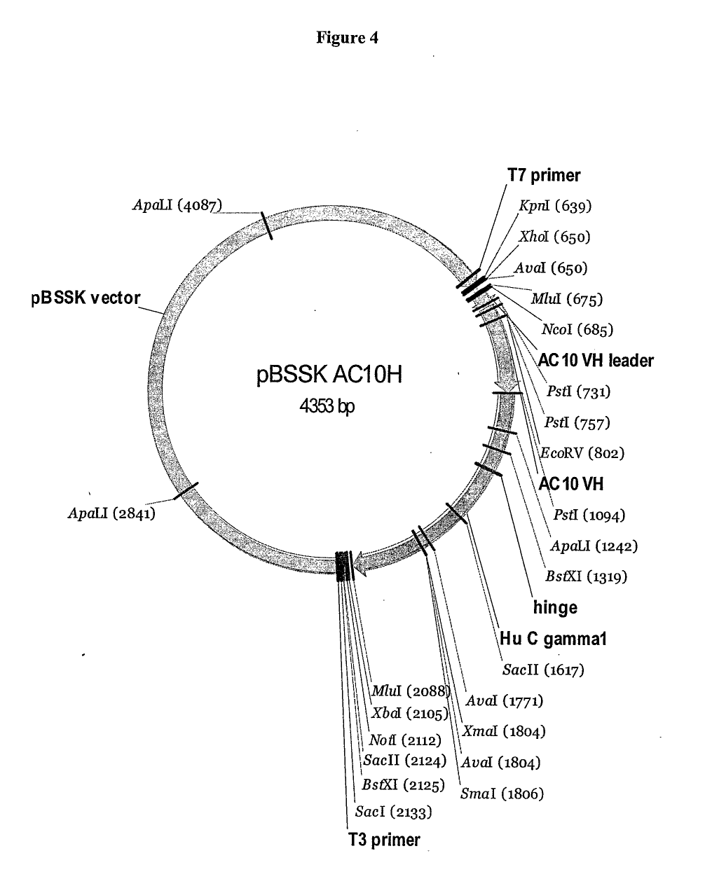

Mutants of cAC10 were generated in pBluescript vectors containing cDNAs for either cAC10 heavy (SEQ ID NO:6) (in pBSSK-AC10H) or cAC10 light (SEQ ID NO:8) (in pBSSK-AC10L) chain and encoding the cAC10 heavy (SEQ ID NO:7) or cAC10 light (SEQ ID NO:9) chain, respectively. Mutagenesis was performed using the Quikchange® Site-Directed Mutagenesis Kit (Stratagene, La Jolla, Calif.) according to the manufacturer's instructions. A pBluescript vector containing the cAC10 heavy chain cDNA, pBSSK AC10H shown in FIG. 4, was used as a template to generate heavy chain C226S, C229S double mutant (having cysteine to serine substitutions are positions 226 and 229). (Residue numbering is of the mature cAC10 heavy and light chains, excluding the signal sequences.) Primer C226S:C229S: 5′GACAAAACTCACACATCCCCACCGTCCCCAGCACCTGAACTC (SEQ ID NO:1) and its reverse complement partner were used to introduce the amino acid substitutions (the mutated codons are underlined). The resulting plasmid was called pBSSK AC10H226,229, containing the cDNA for cAC10H226/229 (SEQ ID NO: 14) and encoding the cAC10 heavy chain C226S, C229S double mutant (SEQ ID NO:15).

A cAC10 heavy chain C220S mutant was generated using pBSSK AC10H as a template and primer C220S: 5′GTTGAGCCCAAATCTTCTGACAAAACTCA-CACATGCCC (SEQ ID NO:2) and its reverse complement partner (the mutated codon is underlined) to produce construct pBSSK AC10H220 containing the cDNA for cAC10 H220 (SEQ ID NO:10) and encoding the cAC10 C220S mutant (SEQ ID NO:11). pBSSK AC10H220 was used as a template to generate heavy chain C220S, C226S double mutant using primer C226S: 5′GACAAAACTCACACATCCCCACCG-TGCCCAGC (SEQ ID NO:3) and its reverse complement partner (the second mutated codon is underlined). The resulting plasmid was called pBSSK AC10H220,226, containing the cDNA for cAC10 H220/226 (SEQ ID NO:12) and encoding the cAC10 heavy chain C220S, C226S double mutant (SEQ ID NO:13). pBSSK AC10 H226,229 was used as a template to generate heavy chain C220S, C226S, C229S mutant using primer C220S: 5′GTTGAGCCCAAATCTTCTGACAAAACTCACACATCCCC (SEQ ID NO:4) and its reverse complement partner (the mutated codon is underlined). The resulting plasmid was called pBSSK AC10H220,226,229, containing the cDNA for cAC10H220/226/229 (SEQ ID NO:16) and encoding the cAC10 heavy chain C220S, C226S, C229S mutant (SEQ ID NO:17).

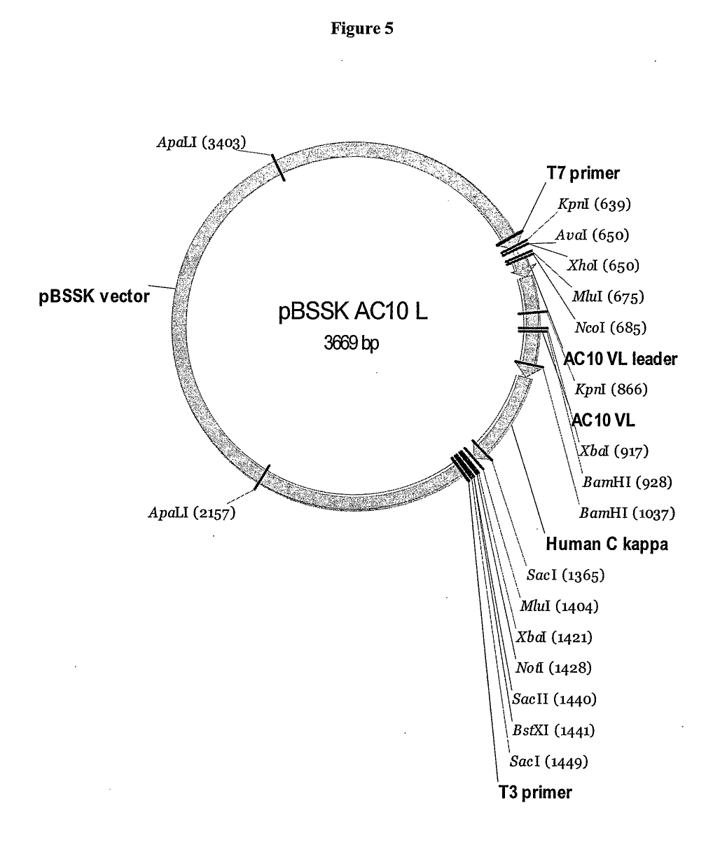

A pBluescript vector containing the cAC10 light chain cDNA, pBSSK AC10L as shown in FIG. 5, was used as a template to generate light chain C218S mutant pBSSK AC10L218 using primer C218S: 5′CTTCAACAGGGGAGAGTCTTAGACGCG-TATTGG (SEQ ID NO:5) and its reverse complement partner (the mutated codon is underlined). The resulting plasmid was called pBSSK AC10L218, containing the cDNA for cAC10 L218 (SEQ ID NO:18) and encoding the cAC10 light chain C218S mutant (SEQ ID NO:19).

cAC10 heavy chain parent and cysteine variant cDNAs were released from pBluescript by cleavage with restriction enzymes XhoI and XbaI and ligated into the pDEF38 expression vector (Running Deer and Allison, Biotechnol Prog. 20(3):880-9 (2004)) downstream of the CHEF EF-1α promoter. cAC10 light chain parent and cysteine variant cDNAs were released from pBluescript with MluI and cloned into the MluI site of pDEF38 downstream of the CHEF EF-1α promoter.

(ii) Stable Cell Line Development and Protein Expression

The cAC10 variants were stably expressed in a CHO-DG44 cell line as previously described for the cAC10 parent antibody (Wahl et al., Cancer Res. 62:3736-3742 (2002)). pDEF38 expression constructs were linearized with restriction enzyme PvuI prior to transfection. Fifty micrograms of linearized pDEF38 cAC10 H chain parent or the cysteine variant construct was cotransfected with 50 μg of linearized pDEF38 cAC10 L chain parent or the cysteine variant construct into CHO-DG44 cells (Urlaub et al., Somat Cell Mol. Genet. 12(6):555-66 (1986)) by electroporation. Following electroporation, the cells were allowed to recover for 2 days in EX-CELL 325 PF CHO medium containing hypoxanthine and thymidine (JRH Bioscience, Lenexa, Kans.) and 4 mM L-glutamine (Invitrogen, Carlsbad, Calif.). After 2 days, stable cell lines expressing the cAC10 variants were selected by replacing the medium with selective medium without hypoxanthine and thymidine. Only cells that incorporated the plasmid DNA, which includes the selectable marker, were able to grow in the absence of hypoxanthine and thymidine. After cells were recovered, stable pools were scaled up to 30 ml shake flask cultures. Cell cloning was performed using a limited dilution method in a background of non-transfected CHO-DG44 feeder cells. Briefly, 0.5 transfected cells and 1000 non-transfected cells were plated per well of a microtiter plate in EX-CELL 325 PF CHO medium in the absence of hypoxanthine and thymidine. Following 7-10 days incubation individual colonies were picked and expanded. High titer clones were selected and cultured in spinners at a final volume of 2.5 L or WAVE bioreactors (WAVE Biotech LLC, Bridgewater, N.J.) at a final volume of 5-10 L.

### Results

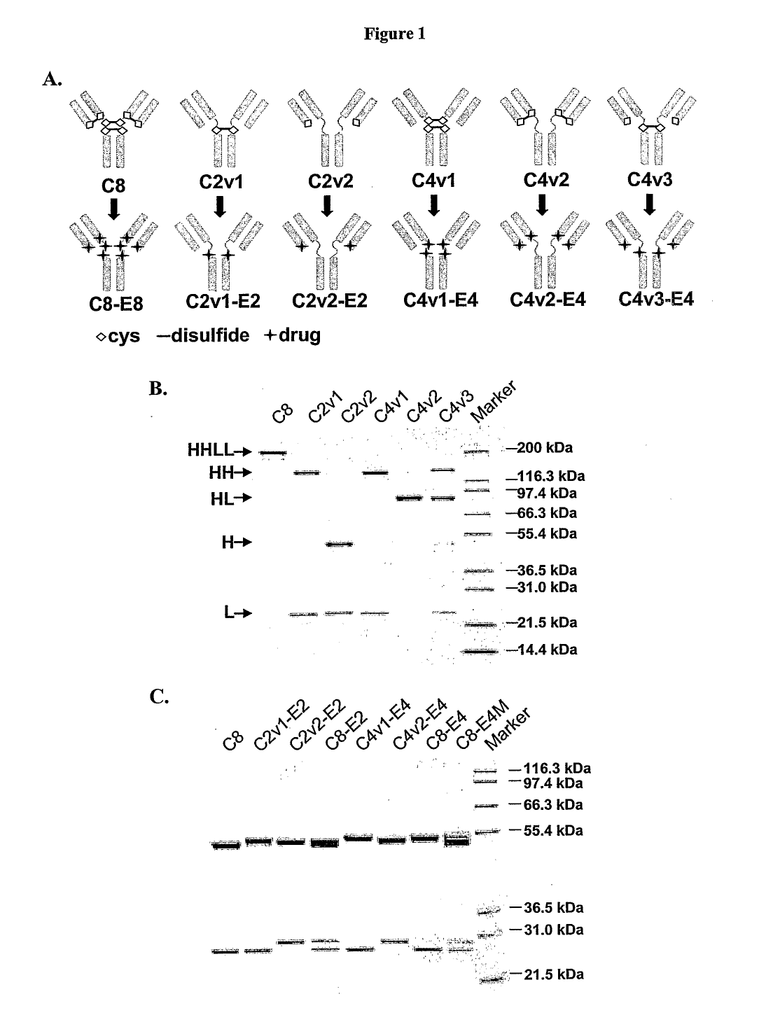

cAC10, is a chimeric IgG₁ that binds to human CD30 (Wahl et al., supra). Antibody cAC10, has 4 solvent accessible inter-chain disulfide bonds that are readily reducible and conjugated to vcMMAE, a thiol-reactive auristatin drug in near quantitative yield (Doronina et al., Nat. Biotechnol. 21:778-784 (2003)). This ADC comprising the cAC10 parent antibody with all 8 accessible cysteines and loaded with vcMMAE is designated here as C8-E8 (FIG. 1A). The accessible cysteines in cAC10 were systematically mutated to a homologous residue, serine, to generate antibody variants with either 4 (C4v1, C4v2 and C4v3) or 2 (C2v1 and C2v2) remaining accessible cysteines (Table 1 and FIG. 1A). In addition, antibody variant C6v1 with heavy chain cysteine residue 226 changed to serine (not shown) had six accessible cysteines. These engineered antibody variants provided a starting point to create conjugates with precisely defined stoichiometry and site of drug attachment.

All antibody variants were expressed in stable CHO-DG44 cell lines at titers of 25-125 mg/L. The antibody variants were purified from 2.5 to 10 L cultures by protein A affinity and ion exchange chromatography (Table 1) and then analyzed by size exclusion chromatography and SDS-PAGE. All antibody variants, except C4v3, were estimated to be >98% monomeric by size exclusion chromatography (Table 2). All variants electrophoresed under reducing conditions gave rise to two major bands consistent with the presence of heavy and light chains (data not shown). As for SDS-PAGE under non-reducing conditions, all antibody variants (except C4v3) gave electrophoretic patterns (FIG. 1B) consistent with the anticipated inter-chain disulfide bonding pattern (FIG. 1A). Antibody variant C4v3 was excluded from the remainder of these studies on the basis of its unanticipated electrophoretic behavior and a size exclusion chromatography profile that suggested significant aggregation.

TABLE 1
Generation and Characterization of Antibody Variants

| | | Competition |
|---|---|---|
| | | binding to |
| cAC10 | Location of | Karpas-299 |
| variant* | Cys→Ser mutations^{†} | (IC₅₀, nM)# |
| C8 | none (parent) | 2.8 ± 0.1 |
| C2v1 | L214, H220, H226 | 2.2 ± 0.4 |
| C2v2 | H220, H226, H229 | 2.6 ± 0.4 |
| C4v1 | L214, H220 | 3.2 ± 0.4 |
| C4v2 | H226, H229 | 2.4 ± 0.1 |
| C4v3 | H220, H226 | nd |

*cAC10 variants are identified by the number of solvent accessible cysteine residues and, where necessary, a variant number. E.g., C2v1 denotes a cAC10 variant containing 2 solvent accessible cysteine residues (FIG. 1A).
^{†}L, light chain; H, heavy chain; numbering scheme of Kabat et al. (Sequences of Proteins of Immunological Interest, 5th ed. NIH, Bethesda, MD (1991)).
#Mean (± SEM) for ≧3 independent experiments.
^{||}nd, not determined due to presence of shoulder and broadening of peak

TABLE 2
Protein recovery for each cAC10 cysteine variant following
Protein A purification and results from size exclusion
chromatography analysis.

| cAC10 cys | Protein recovery in mg/L | |
|---|---|---|
| variant | culture (Protein A purified) | % monomer |
| C2v1 | 120 | 98.8% |
| C2v2 | 92 | 99.1% |
| C4v1 | 33 | 99.5% |
| C4v2 | 36 | 94.6% |
| C4v3 | 37 | * |
| C6v1 | 28 | 97.3% |

* not determined due to presence of shoulder and broadening of peak.

Purified proteins were analyzed by SDS-PAGE under reducing and non-reducing conditions. All variants except cAC10 C4v3 displayed the expected banding pattern under non-reducing conditions as shown in Table 3 and FIG. 1B.

TABLE 3
Expected band patterns and molecular weights for variants
analyzed under non-reducing conditions by SDS-PAGE

| cAC10 | Cysteine | Non-reduced | |
|---|---|---|---|
| Variant | mutations | band pattern | MWs (kDa) |
| C2v1 | L218, H220/226 | HH + L | 98 + 24 |
| C2v2 | H220/226/229 | H + L | 49 + 24 |
| C4v1 | L218, H220 | HH + L | 98 + 24 |
| C4v2 | H226/229 | HL | 73 |
| C4v3 | H220/226 | HH + L | 98 + 24 |
| C6v1 | H226 | HHLL | 146 |

Aggregation was assessed by size exclusion high-performance liquid chromatography and all variants except cAC10 C4v3 were determined to be >94% monomeric. cAC10 C4v3 was found to be heterogeneous by both non-reducing SDS-PAGE and size exclusion analysis. The banding pattern of cAC10 C4v3 under non-reducing conditions included the expected heavy-heavy chain dimer and light chain bands as shown in Table 3 but also a heavy-light chain dimer and heavy chain alone suggesting that the free light chain cysteine was capable of forming a disulfide bond with the heavy chain cysteine at position H229.

### Preparation and Analysis of Antibody Drug Conjugates Procedures

(i) Preparation of Antibody Drug Conjugates

cAC10 parent and cysteine variant antibodies were purified using protein A chromatography and analyzed by SDS-PAGE and size exclusion chromatography. All cAC10 cysteine variants except cAC10 C6v1 were reduced using 10 mM dithiothreitol (DTT; Sigma, St Louis, Mo.), which was an excess over antibody of approximately 100×, in 0.025 M sodium borate pH 8, 0.025 M NaCl, and 1 mM diethylenetriaminepentaacetic acid (DTPA; Aldrich, Milwaukee, Wis.) for 1 h at 37° C. The reduced antibody was diluted to 150 mL with water and applied to a 70 mL hydroxyapatite column (Macroprep ceramic type 140 μm, BioRad) at a flow rate of 10 mL/min. The column was previously equilibrated with 5 column volumes of 0.5 M sodium phosphate pH 7, 10 mM NaCl and 5 column volumes of 10 mM sodium phosphate pH 7, 10 mM NaCl. Following application, the column was washed with 5 column volumes of 10 mM sodium phosphate pH 7, 10 mM NaCl and then eluted with 100 mM sodium phosphate pH 7, 10 mM NaCl. Reduced antibody was titrated with 5,5′-dithio-bis-(2-nitrobenzoic acid) (DTNB; Pierce) to determine the concentration of antibody-cysteine thiols. Reduced antibody was cooled to 0° C. and treated with 2.75 equivalents of maleimidocaproyl-valine-citrulline-p-aminobenzoyl-MMAE (vcMMAE) in DMSO. The final DMSO concentration was 10% to ensure that the drug was fully soluble. After 40 min at 0° C., excess cysteine was added to quench any unreacted vcMMAE and the mixture was diluted to 250 mL with water. The conjugate was purified on a hydroxyapatite column as described above. Antibody-drug conjugates were concentrated and the buffer changed to PBS using 15 mL Amicon Ultrafree 30K cutoff spin concentration devices. cAC10 C6v1 was reduced with a limited number of equivalents of TCEP (tris(2-carboxyethyl)phosphine, Acros) and conjugated to vcMMAE without removal of excess TCEP as follows: 35 mL of C6v1 (2.1 mg/mL or 14.3 μM; 74 mg) were treated with 4.0 equivalents of TCEP (57.1 μM, from 100 mM stock) in PBS with 1 mM DTPA for 2.5 h at 37° C.

The extent of reduction was checked by purifying a small amount of the reduction reaction through a PD-10 column (Amersham Biosciences) and titrating the number of antibody-cysteine thiols with DTNB, yielding 5.7 per C6v1 The reduced antibody was then cooled to 0° C. and treated with 8.0 equivalents of vcMMAE (the concentration of antibody thiols was 73.5 μM and the vcMMAE concentration was 103.2 μM) in 3.9 mL of DMSO. After 135 min at 0° C., 0.4 mL of 100 mM cysteine was added to quench any unreacted vcMMAE and the mixture was diluted to 250 mL with water. The conjugate was purified on a hydroxyapatite column as described above. cAC10-C6v1-vcMMAE (20 mL of 2.4 mg/mL; 48 mg) (C6v1-E6) was concentrated and the buffer changed to PBS using 15 mL Amicon Ultrafree 30K cutoff spin concentration devices.

The generation of parent cAC10 antibody drug conjugates (ADCs) with two and four MMAE molecules per antibody, C8-E2 and C8-E4 respectively, has been described (Hamblett et al., Clin. Cancer Res. 15 7063-7070 (2004)). Briefly, the method involved a partial reduction of the mAb to expose ˜4 reduced Cys per Ab followed by reaction with vc-MMAE. Partially loaded cAC10-Val-Cit-MMAE referred to as C8-E4-Mixture (or C8-E4M) was obtained.

C8-E2 and C8-E4 were prepared from C8-E4M by preparative HIC (hydrophobic interaction chromatography) fractionation on a Toyopearl Phenyl-650M HIC resin (Tosoh Bioscience, Montgomeryville, Pa.) equilibrated with >5 column volumes of Buffer A (50 mM sodium phosphate, 2 M NaCl, pH 7.0). To prepare the sample for loading onto the column, 39 ml of the C8-E4-Mixture (12.9 mg/ml) was mixed with an equivalent volume of Buffer A′ (50 mM sodium phosphate, 4 M NaCl, pH 7.0). Following sample loading, the column was washed with Buffer A until an A₂₈₀ baseline was achieved. C8-E2 was eluted and collected with a step gradient consisting of 65% Buffer A/35% Buffer B (80% v/v 50 mM sodium phosphate, pH 7.0, 20% v/v acetonitrile). After baseline was again achieved, C8-E4 was eluted and collected with a step gradient consisting of 30% Buffer A/70% Buffer B. Both C8-E2 and C8-E4 peaks were collected to ˜20% of their respective peak heights. The fractions of interest were buffer exchanged into PBS using Ultrafree-15 centrifugal filter devices with a molecular weight cutoff of 30 kDa (Millipore, Billerica, Mass.).

(ii) Analysis of Drug Loading

Drug loading was determined by measuring the ratio of the absorbance at 250 and 280 nm (A250/280). The number of vcMMAE per cAC10 was empirically determined to be (A250/A280-0.36)/0.0686. ADCs were analyzed by hydrophobic interaction chromatography (HIC) using a Tosoh Bioscience Ether-5PW column (part 08641) at a flow rate of 1 mL/min and a column temperature of 30° C. Solvent A was 50 mM sodium phosphate pH 7 and 2.5 M NaCl. Solvent B was 80% 50 mM sodium phosphate pH 7, 10% 2-propanol, and 10% acetonitrile. Isocratic 0% B for 15 min, a 50-min linear gradient from 0 to 100% B, a 0.1-min linear gradient from 100 to 0% B, and isocratic 0% B for 14.9 min. Injections (typically 90-100 μL) were 1 volume of ADC (concentration of at least 3 mg/mL) and 1 volume of 5 M NaCl.

ADCs from HIC chromatography were analyzed using an Agilent Bioanalyzer. A protein 200 chip was used under denaturing but nonreducing conditions as described by the manufacturer. Briefly, 4 μL of 1 mg/mL ADC were mixed with 2 μL of nonreducing loading buffer and heated to 100° C. for 5 min. Water (84 μL) was added and 6 μL of this mixture was loaded into each well of the chip.

ADCs were analyzed on a PLRP-S column (Polymer Laboratories part 1912-1802: 1000 A, 8 u, 2.1×50 mm). The flow rate was 1 mL/min and the column temperature was 80° C. Solvent A was 0.05% trifluoroacetic acid in water and solvent B was 0.04% trifluoroacetic acid in acetonitrile. Isocratic 25% B for 3 min, a 15-min linear gradient to 50% B. a 2-min linear gradient to 95% B, a 1-min linear gradient to 25% B, and isocratic 25% B for 2 min. Injections were 10-20 μL of 1 mg/mL ADC previously reduced with 20 mM DTT at 37° C. for 15 min to cleave the remaining interchain disulfides.

### Results

MMAE conjugates of cAC10 cysteine variants were generated by reduction of the antibody followed by alkylation with vcMMAE. Analysis of each conjugated cAC10 cysteine variant by both UV-VIS analysis at an absorbance of 280 nm and PLRP chromatography demonstrated that close to the expected drug loading was achieved as shown in Table 4.

TABLE 4
Summary of the analysis of cAC10 cysteine variant vcMMAE conjugates

| cAC10 | Conc. | | Total | DTNB | PLRP | % | |
|---|---|---|---|---|---|---|---|
| Variant | (mg/ml) | Volume (ml) | (mg) | RSH/Ab | Drug/ab | monomer | Free drug |
| C2v1-E2 | 41 | 2.3 | 94.3 | 2.3 | 2.1 | 98.0% | <0.05% |
| C2v2-E2 | 5.6 | 0.6 | 3.4 | 2.3 | 2.0 | 99.4% | <0.05% |
| C4v1-E4 | 12.0 | 3.8 | 45.6 | 4.2 | 3.7 | 99.1% | <0.05% |
| C4v2-E4 | 12.4 | 4.0 | 49.6 | 4.0 | 3.5 | 98.7% | <0.05% |
| C4v3-E4 | 0.8 | 0.4 | 0.3 | 7.2 | 3.7 | 99% | <0.05% |
| C6v1-E6 | 12.5 | 3.3 | 40.6 | 5.7 | 5.7 | 98.7% | <0.05% |

Analysis by size exclusion chromatography demonstrated that all conjugates consisted of 98% monomer or greater as shown in Table 4. The control molecules described in this study were parent cAC10 conjugated with either two molecules of MMAE (C8-E2), or four molecules of MMAE (C8-E4). These two and four drug-loaded MMAE conjugates were generated by partial reduction of the parent cAC10 antibody and analyzed as previously described in Hamblett et al., Clin. Cancer Res. 15:7063-7070 (2004).

In Vitro Cytotoxicity of cAC10 Cysteine Variant Conjugates

### Procedures

Growth inhibition of CD 30⁺ Karpas 299 cells treated with cAC10 cysteine variant conjugates was determined by measuring DNA synthesis. Conjugates were incubated with cells for 92 hours followed by labeling with [³H]-thymidine, 0.5 μCi/well for 4 hours at 37° C. Cells were harvested onto filters and mixed with scintillation fluid and radioactivity was measured using a Topcount Scintillation counter (Packard Instruments, Meriden, Conn.). The percent radioactivity incorporated relative to the untreated controls was plotted versus conjugate concentration and the data were fit to a sigmoidal dose-response curve using Prism 4 software (GraphPad Software Inc, San Diego, Calif.). Alternatively, 50 μM resazurin was added to Karpas 299 cells following the 92 hour incubation period with conjugate. After a 4 hour incubation period dye reduction was measured using a Fusion HT fluorescent plate reader (Packard Instruments, Meriden, Conn.).

### Results

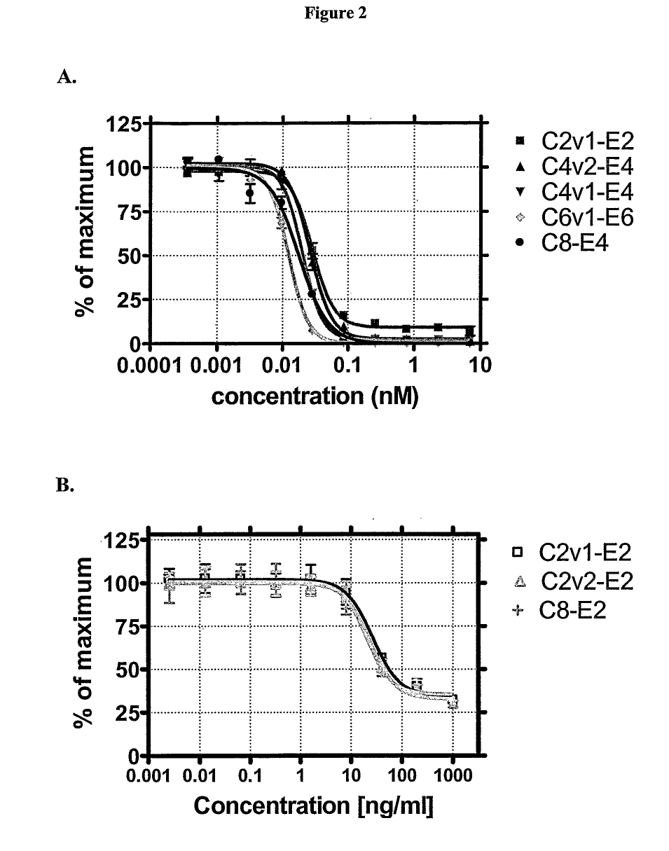

The cytotoxicities of the AC10 cysteine variant C2v1, C4v1, C4v2, and C6v1 MMAE conjugates (C2v1-E2, C4v1-E4, C4v2-E4, and C6v1-E6, respectively) were tested using a [³H]-thymidine incorporation assay on CD30⁺ Karpas 299 cells. The control conjugate used was the four drug-loaded parent cAC10 (C8-E4) which has been shown to have potency that lies between the fully loaded parent cAC10 MMAE conjugate (C8-E8) (which is the most potent), and the two-drug loaded conjugate (C8-E2). C6v1-E6 had the lowest IC₅₀ value of 0.012 nM, while the four drug-loaded cysteine variants C4v1-E4 and C4v2-E4 and the four drug-loaded parent cAC10 conjugate C8-E4 had very similar IC₅₀s of 0.020 nM, 0.027 nM and 0.018 nM, respectively, as shown in FIG. 2A. As shown in FIG. 2B, the C2v1-E2 MMAE conjugate had an IC₅₀ of 0.029 DM. Subsequently, the in vitro cytotoxic activities of both C2v1-E2 and C2v2-E2 MMAE drug conjugates on Karpas 299 cells were evaluated. Cytotoxicity was assessed by reduction of resazurin dye which was introduced to the culture following 92 hours continuous exposure to conjugate. cAC10 conjugated with two MMAE drug molecules per antibody (C8-E2) was used as the control. All three conjugates had similar IC₅₀ values of 52.4 ng/ml, 39.8 ng/ml and 39.8 ng/ml for C2v1-E2, C2v2-E2 and C8-E2, respectively. These data demonstrate that the cysteine variant conjugates compare closely in activity to partially loaded MMAE conjugates generated from the parent cAC10 antibody by partial reduction.

Antitumor Activity of cAC10 Cysteine Variant Conjugates In Vivo Using a Xenograft Model of Human ALCL

### Procedures

To establish a subcutaneous disease model of ALCL 5×10⁶ Karpas-299 cells were implanted into the right flank of C.B-17 SCID mice (Harlan, Indianapolis, Ind.). Therapy with ADCs was initiated when the tumor size in each group of 6-10 animals averaged 100 mm³. Treatment consisted of a single injection. Tumor volume was calculated using the formula (Iength×width²)/2. A tumor that decreased in size such that it was impalpable was defined as a complete regression (CR). A complete regression that lasted beyond 100 days post tumor implant was defined as a cure. Animals were euthanized when tumor volumes reached approximately 1000 mm³.

### Results

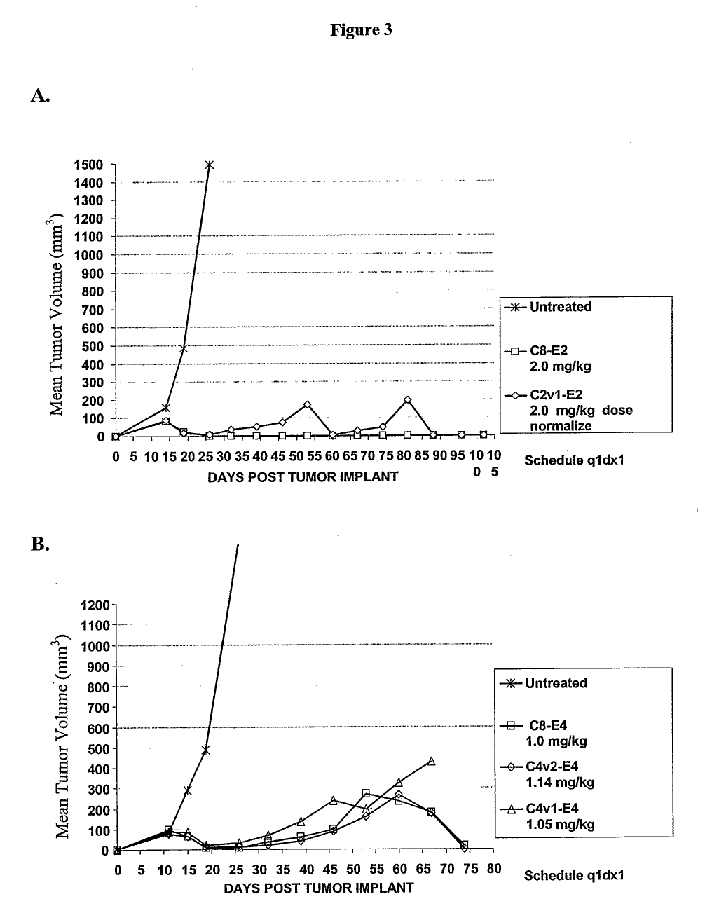

The efficacies of the cAC10 cysteine variant drug conjugates were assessed in a subcutaneous xenograft model of ALCL in SCID mice. Karpas 299 cells were implanted into the flanks of SCID mice and tumors were grown to an average volume of 100 mm³. Tumor bearing mice were randomly divided into groups of eight to ten animals and either left untreated or were treated with cAC10 cysteine variant MMAE conjugates C2v1-E2, C4v1-E4 or C4v2-E4 or partially MMAE loaded parent cAC10 conjugates C8-E2 and C8-E4 in a single dose study. ADC doses were normalized so an equal concentration of MMAE was injected per group with 1 mg/kg, 1.14 mg/kg and 1.05 mg/kg injected for C8-E4, C4v2-E4 and C4v1-E4, respectively, and 2 mg/kg and 1.9 mg/kg for C8-E2 and C2v1-E2, respectively. As shown in FIG. 3A, C2v1-E2 showed similar antitumor activity to C8-E2 with complete tumor regressions occurring in all animals treated with C8-E2 and six of eight animals treated with C2v1-E2. As shown in FIG. 3B, C4v1-E4 and C4v2-E4 displayed similar antitumor activities to C8-E4. Complete regressions occurred in eight of ten animals for C8-E4 and C4v2-E4 and six of ten animals for C4v1-E4.

In summary, the two and four drug loaded ADCs generated from the cysteine variants have similar in vivo activity to the C8-E4 and C8-E2 conjugates produced by the partial reduction method.

### Example 2

### Preparation and Analysis of Antibody Conjugates

### Procedures

The cAC10 parent and variant antibodies prepared as described in Example 1 were purified by protein A followed by anion exchange chromatography using an ÄKTAexplorer (GE Healthcare, Piscataway, N.J.). Briefly, the antibody-containing conditioned media were concentrated ˜10-fold and buffer-exchanged into PBS, pH 7.4 by tangential flow filtration (Millipore). The concentrated samples were treated with 0.5% (v/v) Triton X-100 (Sigma, St. Louis, Mo.) with gentle stirring overnight at 4° C. for endotoxin removal, before loading onto protein A (GE Healthcare) pre-equilibrated with PBS, pH 7.4. The column was washed with PBS, pH 7.4, 2-3 column volumes (CV) 0.5% v/v Triton X-100, 1 M NaCl in PBS, pH 7.4 then with PBS, pH 7.4 until a stable baseline was reached. Bound antibody was eluted from protein A with 30 mM sodium acetate, pH 3.6 and then dialyzed against 20 mM Tris-HCl, 10 mM NaCl, 1 mM EDTA, pH 8.0 (buffer A). The pooled antibody was then loaded on to Q sepharose (GE Healthcare) pre-equilibrated with buffer A and washed with 2-3 CV buffer A, 5-10 CV buffer A containing 0.5% (v/v) Triton X-100 with incubation and 5 CV buffer A. Antibodies were eluted from Q sepharose by either step or linear NaCl gradient (from 10-500 mM NaCl in buffer A) and dialyzed against PBS, pH 7.4. Purified antibodies were analyzed by SDS-PAGE and by TSK-Gel G3000SW HPLC size exclusion chromatography (Tosoh Bioscience, Montgomeryville, Pa.).

Conjugation of cAC10 Cys→Ser antibody variants with either 2 (C2v1-E2, C2v2-E2) or four (C4v1-E4, C4v2-E4 and C4v3-E4) equivalents of MMAE molecules involved reduction with a few (2.5 to 4) equivalents of tris(2-carboxyethyl)phosphine (Acros Organics, Geel, Belgium) and conjugation to maleimidocaproyl-valine-citrulline-p-aminobenzoyl-MMAE (vcMMAE) (Doronina et al., supra) without removal of excess tris(2-carboxyethyl)phosphine. Prior to drug addition the extent of reduction was assessed by purifying a small amount of the reduction reaction through a PD-10 column (GE Healthcare) and titrating the number of antibody-cysteine thiols with 5,5′-dithio-bis(2-nitrobenzoic acid) (Ellman, Arch. Biochem. Biophys. 74:443-450 (1958)). The reduced antibodies were reacted with vcMMAE for 60 min at 0° C. and excess N-acetylcysteine (Acros Organics) was then added to quench any unreacted maleimidocaproyl-Val-Cit-MMAE. The reaction mixture was then diluted 5-fold with water and then loaded on to hydroxyapatite column equilibrated with 10 mM sodium phosphate pH 7.0, 10 mM NaCl. The column was washed with 5 CV of the same buffer and the conjugate eluted with 100 mM sodium phosphate pH 7.0, 10 mM NaCl. The conjugates were concentrated and buffer-exchanged into PBS using Amicon Ultrafree centrifugal filter units (Millipore).

The generation of parent cAC10 conjugates with a mean stoichiometry of 4 drugs per antibody (range of 0 to 8 drugs), C8-E4 mixture (C8-E4M), and 2 drugs per antibody, C8-E2M, have been described (Hamblett et al., supra) (Sun et al., Bioconjug. Chem. 16:1282-1290 (2005)). C8-E2M was subjected to hydrophobic interaction chromatography to isolate conjugates loaded with 4 (C8-E4) and 2 (C8-E2) MMAE molecules per antibody, as previously described (Hamblett et al., supra).

ADCs were analyzed to determine the stoichiometry of drug loading using the molar extinction coefficients at wavelengths of 248 nm and 280 nm for the antibody (9.41×10⁴ and 2.34×10⁵ M⁻¹ cm⁻¹, respectively) and drug (1.50×10³ and 1.59×10⁴ M⁻¹ cm⁻¹, respectively), as previously described (Hamblett et al., supra). The location of drug attachment to the antibody heavy and light chains was investigated by reverse phase HPLC using a PLRP-S column (Polymer Laboratories, Amherst, Mass.; #1912-1802: 1000 Å, 8 μm, 2.1×150 mm) and solvents A (0.05% (v/v) trifluoroacetic acid in water) and solvent B (0.04% (v/v) trifluoroacetic acid in acetonitrile). The running conditions (1 ml/min, 80° C.) were: isocratic 25% solvent B (3 min), linear gradient to 50% solvent B (25 min), linear gradient to 95% solvent B (2 min), linear gradient to 25% solvent B (1 min), and isocratic 25% solvent B for 2 min. Prior to chromatography ADC samples (10-20 μl, 1 mg/ml) were reduced with 20 mM DTT at 37° C. for 15 min to cleave the remaining inter-chain disulfide bonds.

### Results

The cAC10 parent antibody (C8) was partially reduced to yield a mean of 2 or 4 sulfhydryl groups per antibody and then reacted with vcMMAE. The corresponding conjugation products, C8-E2M and C8-E4M, have a mean loading of 2 and 4 equivalents of MMAE respectively. C8-E2M and C8-E4M are mixtures of species loaded with 0, 2, 4, 6 or 8 equivalents of MMAE per antibody (Hamblett et al., supra). Conjugates with uniform stoichiometry of either 2 (C8-E2), or 4 (C8-E4) equivalents of MMAE were purified from the C8-E2M mixture by hydrophobic interaction chromatography as previously described (Hamblett et al., supra). MMAE conjugates of the engineered antibody variants were generated by reduction of the antibody followed by reaction with vcMMAE.

For each ADC, the observed drug loading stoichiometry by spectrophotometric (Hamblett et al., supra) and reverse phase HPLC analyses (Sun et al., supra) closely matched those expected. Peak area analysis following size exclusion chromatography suggested that all ADCs were >98% monomeric (Doronina et al., supra). The yield of the Cys→Ser variant conjugates (89-96%) was greatly improved compared to the conjugates C8-E4 (11%) and C8-E2 (27%) purified from C8-E2M. SDS-PAGE analysis of the ADCs under reducing conditions showed the expected reduced motility of the MMAE conjugated light chains in C2v2-E2, C8-E2, C4v2-E4, C8-E4 and C8-E4M compared to the unconjugated light chains in the other ADCs. The decreased motility of the conjugated heavy chains was less pronounced but the heterogeneity of conjugated heavy chains in C8-E2 and C8-E4M was apparent (FIG. 1C).

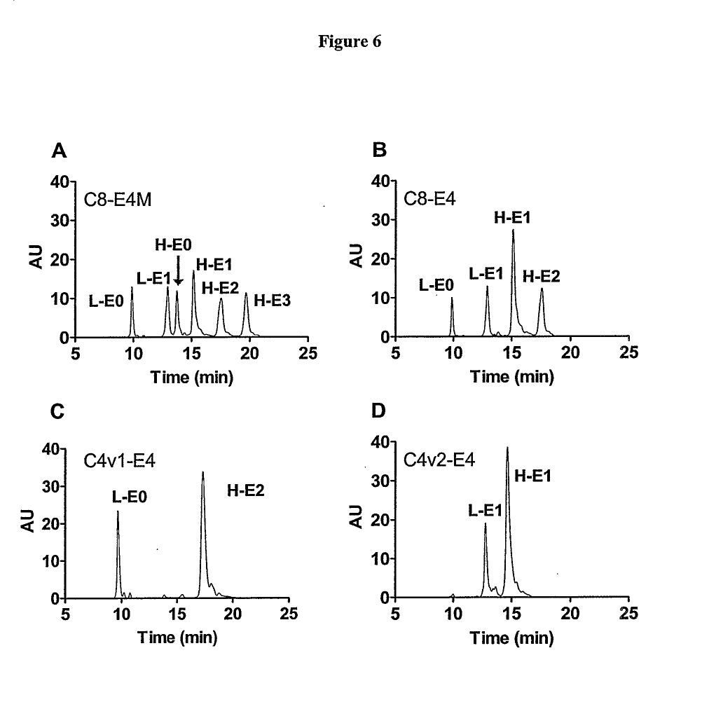

Reverse phase HPLC under reducing conditions was used to evaluate ADC heterogeneity. This method resolves light chains loaded with 0 or 1 equivalents of MMAE (L-E0 and L-E1, respectively) as well as heavy chains loaded with 0, 1, 2, or 3 equivalents of MMAE (H-E0, H-E1, H-E2 and H-E3, respectively). C8-E4M (FIG. 6A) is the most heterogeneous conjugate containing all 6 possible species. Purification of C8-E4M to generate C8-E4 reduces the heterogeneity down to 4 species: L-E0, L-E1, H-E1 and H-E2 (FIG. 6B). The homogeneity of cAC10 Cys→Ser variants is demonstrated by the presence of the anticipated single light and heavy chain peaks, L-E0 plus H-E2, and L-E1 plus H-E1, for C4v1-E4 (FIG. 6C) and C4v2-E4 (FIG. 6D), respectively.

In Vitro Characterization of cAC10 Variants and Drug Conjugates

### Procedures

CD30-positive ALCL line Karpas-299 and CD30-negative WSU-NHL were obtained from the Deutsche Sammlung von Mikroorganism und Zellkulturen GmbH (Braunschweig, Germany). L540cy, a derivative of the HD line L540 adapted to xenograft growth, was developed by Dr. Harald Stein (Institüt für Pathologie, University Veinikum Benjamin Franklin, Berlin, Germany). Cell lines were grown in RPMI-1640 media (Life Technologies, Gaithersburg, Md.) supplemented with 10% fetal bovine serum.

Competition binding of the cAC10 variants and their corresponding ADCs was undertaken to assess the impact of mutations and drug conjugation upon antigen binding. Briefly, CD30-positive Karpas-299 cells were combined with serial dilutions of the cAC10 parent antibody, variants or corresponding ADC (prepared as described in Example 1), in the presence of 1 μg/ml cAC10 labeled with europium (Perkin Elmer, Boston, Mass.) in staining medium (50 mM Tris-HCl pH 8.0, 0.9% NaCl (w/v), 0.5% bovine serum albumin (w/v), 10 μM EDTA) for 30 min on ice then washed twice with ice-cold staining medium. Labeled cells were detected using a Fusion HT microplate reader (Perkin-Elmer). Sample data were baseline-corrected and reported as the percent of maximum fluorescence as calculated by the sample fluorescence divided by the fluorescence of cells stained with 1 μg/ml cAC10-europium alone.

Growth inhibition of CD30-positive Karpas 299 or L540cy cells or CD30-negative WSU-NHL cells treated with cAC10 Cys→Ser variant conjugates was determined by incubating conjugates with cells for 92 h followed by incubation with 50 μM resazurin for 4 h at 37° C. Dye reduction was measured using a Fusion HT microplate reader. Data were analyzed by a non-least squares 4-parameter fit using Prism v4.01 (GraphPad Software Inc, San Diego, Calif.).

### Results

Competition binding experiments revealed that neither Cys Ser mutations (Table 1) nor MMAE conjugation (Table 5) impaired antigen binding. Next the cytotoxicities of the cAC10 Cys→Ser variant conjugates were assessed on CD30 positive (Karpas-299 and L540cy) and negative (WSU-NHL) cell lines. The C2v1-E2 and C2v2-E2 conjugates had very similar potency to C2-E2 on both CD30 positive cell lines (Table 5). Similarly, C4v1-E4, C4v2-E4, C8-E4M and C8-E4 displayed similar activity on both CD30 positive cell lines tested (Table 5). Thus, precisely defining the site of stoichiometry of drug attachment did not significantly impact the cytotoxic activity of the conjugate. Increasing the amount drug loading from 2 to 4 MMAE/Ab increased the potency (reduced IC₅₀ values) consistent with previous observations (Hamblett et al., supra). CD30 negative WSU-NHL cells were insensitive to all cAC10 ADCs.

TABLE 5
Generation and Characterization of Antibody Drug Conjugates

| | | Drugs | | Competition | | | Single |
|---|---|---|---|---|---|---|---|
| cAC10 | Percentage | per IgG: | | binding to | Karpas-299 | L540cy | dose |
| drug | yield of | method 1, | Percentage | Karpas-299 | cytotoxicity | cytotoxicity | MTD |
| conjugate* | conjugate^{†} | method 2^{‡} | monomer^{§} | (IC₅₀, nM)^{#} | (IC₅₀, nM) | (IC₅₀, nM) | (mg/kg) |
| C2v1-E2 | 92.7 | 2.0, 1.9 | 98.4 | 2.9 ± 0.3 | 0.26 ± 0.12 | 0.28 ± 0.03 | 40 |
| C2v2-E2 | 88.9 | 2.1, 1.8 | 98.5 | 2.5 ± 0.1 | 0.46 ± 0.30 | 0.27 ± 0.02 | 60 |
| C8-E2 | 27.4^{\|\|} | 2.0, 2.0 | 99.7 | 2.8 ± 0.2 | 0.32 ± 0.21 | 0.28 ± 0.02 | 40 |
| C8-E2M | 97.3 | 2.0, 2.0 | n/d | n/d | n/d | n/d | n/d |
| C4v1-E4 | 90.6 | 4.0, 3.8 | 99.2 | 3.2 ± 0.1 | 0.07 ± 0.02 | 0.13 ± 0.01 | 20 |
| C4v2-E4 | 96.0 | 4.1, 3.8 | 99.0 | 2.8 ± 0.2 | 0.07 ± 0.02 | 0.12 ± 0.01 | <20 |
| C8-E4 | 10.8^{\|\|} | 4.0, 4.0 | 99.5 | 2.4 ± 0.3 | 0.07 ± 0.01 | 0.18 ± 0.04 | 20 |
| C8-E4M | 95 | 4.4, 4.4 | 98.8 | 3.0 ± 0.4 | 0.03 ± 0.01 | 0.07 ± 0.02 | <20 |

Free drug in all ADC preparations was below the detection limit (<0.05%)
*ADCs are identified by their cAC10 variant name (see Table 1) loading level with the drug, MMAE, and whether the drug stoichiometry is variable (M) or fixed. For example, C8-E4M and C8-E2M denotes the parent antibody, cAC10, loaded with a mean of 4 (range 0 to 8) and 2 (range of 0 to 8) equivalents of MMAE per IgG, respectively. The fixed stoichiometry ADCs, C8-E4 and C8-E2, were obtained by purification of the variable stoichiometry ADC, C8-E2M, by hydrophobic interaction chromatography.
^{†}Yield of conjugate obtained as a percentage of purified antibody.
^{‡}Methods 1 and 2 refer to the ratio of absorbance at wavelengths of 248 nm and 280 nm (Hamblett et al., 2004) and reverse phase HPLC analysis under reducing conditions (FIG. 6).
^{§}Estimated from the peak areas in size exclusion chromatography.
^{||}Percentage yield after hydrophobic interaction chromatography based on starting cAC10 protein.
^{#}Mean (±SEM) for ≧3 independent experiments.

### Antitumor Activity of Antibody Cys→Ser Variant Conjugates In Vivo

### Xenograft Models

5×10⁶ Karpas-299 or L540cy cells were implanted into the right flank of C.B-17 SCID mice (Harlan, Indianapolis, Ind.) to establish a subcutaneous disease model of anaplastic large cell lymphoma or Hodgkin's disease, respectively. Tumor volume was calculated using the formula (A×B²)/2, where A and B are the largest and second largest perpendicular tumor dimensions, respectively. Tumor bearing mice were randomly divided into groups of 8-10 animals when the mean tumor volume was 100 mm³. Mice groups were either left untreated or treated with a single intravenous dose of an ADC. For the L540cy xenograft studies the doses used were 6.0 and 12.0 mg/kg for the 2 drug/Ab conjugates and 3.0 and 6.0 mg/kg for the 4/drug Ab conjugates. For the Karpas 299 xenograft model doses used were 0.5, 1.0 and 2.0 mg/kg for the 2 drug/Ab conjugates and 0.5 and 1.0 mg/kg for the 4 drug/Ab conjugates. A tumor that decreased in size such that it was impalpable was defined as a complete regression. A complete regression that lasted beyond 100 d post tumor implant was defined as a “cure”. Animals were euthanized when tumor volumes reached ˜1000 mm³.

### Results

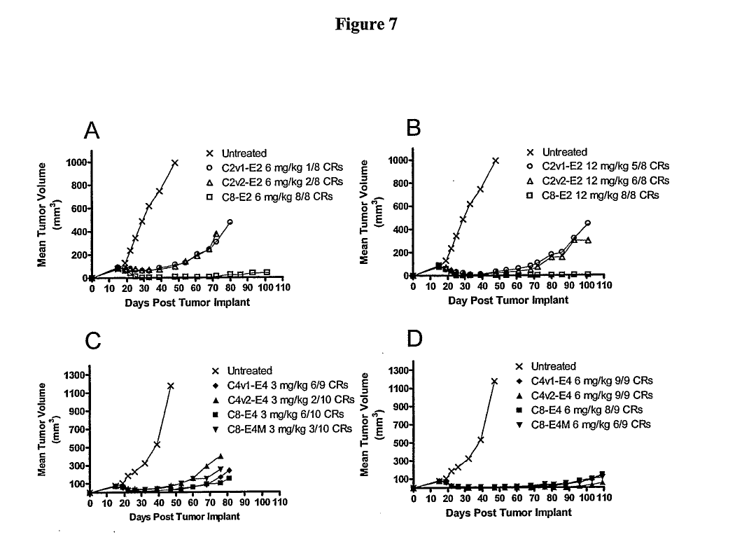

The efficacies of the cAC10 Cys→Ser variant drug conjugates, C2v1-E2, C2v2-E2, C4v1-E4 and C4v2-E4, were compared to conjugates of the parent antibody, C8-E2, C8-E4 and C8-E4M, in subcutaneous xenograft models of anaplastic large cell lymphoma (Karpas-299) and Hodgkin's disease (L540cy) in SCID mice. Briefly, mice bearing 100 mm³ L540cy tumors (mean size) were dosed once with a 2 drug/Ab conjugate (6.0 or 12.0 mg/kg) or a 4 drug/Ab conjugate (3.0 or 6.0 mg/kg) or left untreated. Responses to treatment with C2v1-E2 and C2v2-E2 were comparable and complete regressions were induced at both 6.0 and 12.0 mg/kg doses (FIG. 7A, B). C8-E2 was slightly more potent than C2v1-E2 and C2v2-E2 with cures achieved at both dose levels (FIG. 3A, B). Karpas 299 xenograft models treated with single doses of the 2-drug loaded conjugates showed similar response trends with 3 of the 10 animals achieving complete regressions for C2v1-E2 and C2v2-E2 and 8 of 10 complete regressions for C8-E2 at a 1 mg/kg dose (data not shown). Treatment of L540cy xenograft models with C4v1-E4, C4v2-E4, C8-E4 and C8-E4M resulted in comparable responses with cures achieved at both 3 and 6 mg/kg for each ADC (FIG. 7C, D). Treatment of Karpas 299 models with the 4-drug loaded variants at 0.5 and 1 mg/kg also showed no discrimination between molecules (data not shown).

### Determination and Analysis of Maximum Tolerated Dose

The single dose tolerability of each ADC was determined in Sprague-Dawley rats (Harlan, Ind.). Groups of three rats were injected with 40-80 mg/kg of C2v1-E2, C2v2-E2 and C8-E2 and 20-40 mg/kg of C4v1-E4, C4v2-E4, C8-E4 and C8-E4M via the tail vein to determine the single dose maximum tolerated dose (MTD). Rats were monitored daily for 14 d, and both weight and clinical observations were recorded. Rats that developed significant signs of distress were euthanized. The maximum tolerated dose was defined as the highest dose that did not induce >20% weight loss or severe signs of distress in any of the animals.

For the 2-drug loaded conjugates rats were dosed at 40, 60 and 80 mg/kg. The 40 mg/kg dose was well tolerated while the 60 mg/kg dose was only well tolerated by rats treated with C2v2-E2. One animal injected with 60 mg/kg of C2v1-E2 was sacrificed on day 7 while the remaining 2 animals had a maximum weight loss 6% on day 8 after which weight loss was recovered. One animal dosed with 60 mg/kg of C8-E2 displayed 11% weight loss and was found dead on day 11. The 80 mg/kg dose of each 2-loaded ADC was not well tolerated. Based on these data the MTDs of C2v1-E2, C2v2-E2 and C8-E2 were determined to be 40, 60 and 40 mg/kg, respectively. The 4-drug loaded ADCs were each dosed at 20, 30 and 40 mg/kg. Animals injected with the 20 mg/kg dose of C4v1-E4 and C8-E4 experienced no adverse effects while several animals in the groups treated with the 20 mg/kg doses of C4v2-E4 and C8-E4M showed signs of distress and one from each group was sacrificed on day 9. The higher doses of 30 and 40 mg/kg of each 4-drug loaded ADC were not tolerated. The MTDs for C4v1-E4 and C8-E4 were determined to be 20 mg/kg while the MTDs for C4v2-E4 and C8-E4M were determined to be <20 mg/kg.

No license is expressly or implicitly granted to any patent or patent applications referred to or incorporated herein. The discussion above is descriptive, illustrative and exemplary and is not to be taken as limiting the scope defined by any appended claims.

Various references, including patent applications, patents, and scientific publications, are cited herein, the disclosures of each of which is incorporated herein by reference in its entirety.

1. An immunoconjugate comprising:
an engineered antibody having (a) a functionally active antigen-binding region for a target antigen, (b) at least one interchain cysteine residue, (c) at least one amino acid substitution of an interchain cysteine residue, and (d) a diagnostic, preventative or therapeutic agent conjugated to at least one interchain cysteine residue.

2. The immunoconjugate of claim 1, having four interchain cysteine residues and four amino acid substitutions of interchain cysteine residues.

3. The immunoconjugate of claim 1, comprising two interchain cysteine residues and six amino acid substitutions of interchain cysteine residues.

4. The immunoconjugate of claim 1, which is an IgG1 or an IgG4.

5. The immunoconjugate of claim 1, wherein each the amino acid substitutions is a cysteine to serine substitution.

6. The immunoconjugate of claim 1, wherein the diagnostic, preventative or therapeutic agent is a therapeutic agent.

7. The immunoconjugate of claim 6, wherein the therapeutic agent is an auristatin or an auristatin derivative.

8. The immunoconjugate of claim 7, wherein the auristatin derivative is dovaline-valine-dolaisoleunine-dolaproine-phenylalanine (MMAF) or monomethyauristatin E (MMAE).

9. The immunoconjugate of claim 1, wherein the diagnostic, preventative or therapeutic agent is a diagnostic agent.

10. The immunoconjugate of claim 9, wherein the diagnostic agent is a radioactive agent, an enzyme, a fluorescent compound or an electron transfer agent.

11. The immunoconjugate of claim 1, wherein the antibody binds to CD20, CD30, CD33, CD40, CD70 or Lewis Y.

12. The immunoconjugate of claim 1, wherein the antibody binds to an immunoglobulin gene superfamily member, a TNF receptor superfamily member, an integrin, a cytokine receptor, a chemokine receptor, a major histocompatibility protein, a lectin, or a complement control protein.

13. The immunoconjugate of claim 1, wherein the antibody binds to a microbial antigen.

14. The immunoconjugate of claim 1, wherein the antibody binds to a viral antigen.

15. The immunoconjugate of claim 1, wherein the antibody is an anti-nuclear antibody, anti-ds DNA antibody, anti-ss DNA antibody, anti-cardiolipin antibody IgM or IgG, anti-phospholipid antibody IgM or IgG, anti-SM antibody, anti-mitochondrial antibody, anti-thyroid antibody, anti-microsomal antibody, anti-thyroglobulin antibody, anti-SCL 70 antibody, anti-Jo antibody, anti-U1RNP antibody, anti-La/SSB antibody, anti-SSA antibody, anti-SSB antibody, anti-perital cells antibody, anti-histone antibody, anti-RNP antibody, anti-C ANCA antibody, anti-P ANCA antibody, anti-centromere antibody, anti-fibrillarin antibody, or anti-GBM antibody.

16. The immunoconjugate of claim 1, wherein the antibody is an antibody fragment.

17. The immunoconjugate of claim 16, wherein the antibody fragment is selected from Fab, Fab′ and scFvFc.

18. The immunoconjugate of claim 17, wherein the fragment is an Fab′ or an scFvFc.

19. The immunoconjugate of claim 1, having the following formula:
Ab_{z}Aₐ-W_{w}-Y_{y}-D)ₚ
or a pharmaceutically acceptable salt or solvate thereof,
wherein:
Ab is an antibody,
A is a stretcher unit,
a is 0 or 1,
each W is independently a linker unit,
w is an integer ranging from 0 to 12,
Y is a spacer unit, and
y is 0, 1 or 2,
p ranges from 1 to about 20, and
D is a diagnostic, preventative and therapeutic agent, and
z is the number of predetermined conjugation sites on the protein.

20. The immunoconjugate of claim 19, having the formula: 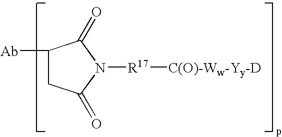
wherein R¹⁷ is selected from —C₁-C₁₀ alkylene-, —C₃-C₈ carbocyclo-, —O—(C₁-C₈ alkyl)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —(CH₂CH₂O)ᵣ—, and —(CH₂CH₂O)ᵣ—CH₂—.

21. The immunoconjugate according to claim 19, having the following formula: 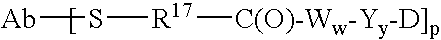
wherein R¹⁷ is selected from —C₁-C₁₀ alkylene-, —C₃-C₈ carbocyclo-, —O—(C₁-C₈ alkyl)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀-alkylene-(C₃-C₈ heterocyclo)-, —(C₃-C₈ heterocyclo)-C₁-C₁₀ alkylene-, —(CH₂CH₂O)ᵣ—, and —(CH₂CH₂O)ᵣ—CH₂—.

22. The immunoconjugate of claim 19, having the formula:

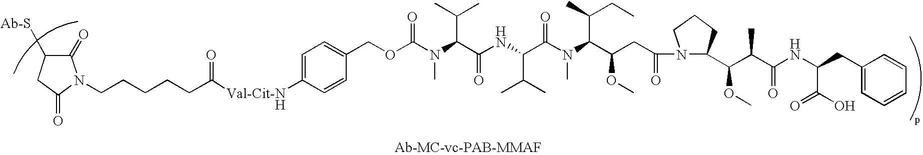

23. The immunoconjugate of claim 19, having the formula:

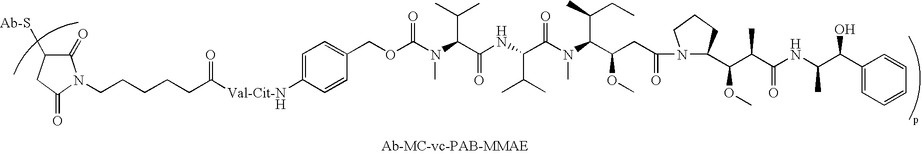

24. The immunoconjugate of claim 19, having the formula:

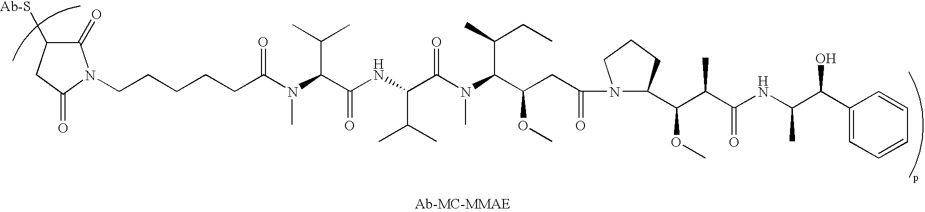

25. The immunoconjugate of claim 19, having the formula:

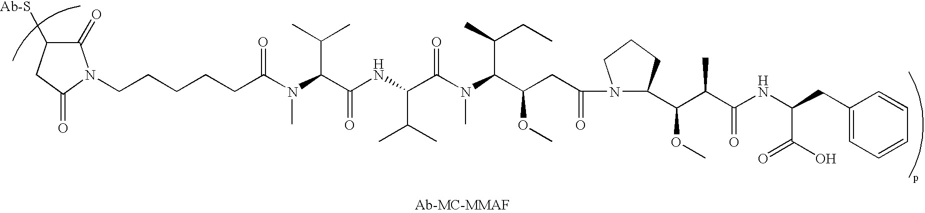

26. A pharmaceutical composition comprising the immunoconjugate of claim 1 and a pharmaceutically acceptable carrier.

27. The pharmaceutical composition of claim 26, wherein the immunoconjugate is formulated with a pharmaceutically acceptable parenteral vehicle.

28. The pharmaceutical composition of claim 26, wherein the immunoconjugate is formulated in a unit dosage injectable form.

29. A method for killing or inhibiting the proliferation of tumor cells or cancer cells comprising treating tumor cells or cancer cells with an amount of the immunoconjugate of claim 6, or a pharmaceutically acceptable salt or solvate thereof, being effective to kill or inhibit the proliferation of the tumor cells or cancer cells.

30. A method for treating cancer comprising administering to a patient an amount of the immunoconjugate of claim 6 or a pharmaceutically acceptable salt or solvate thereof, said amount being effective to treat cancer.

31. A method for treating an autoimmune disease, comprising administering to a patient an amount of the immunoconjugate of claim 6 or a pharmaceutically acceptable salt or solvate thereof, the amount being effective to treat the autoimmune disease.

32. A method for treating an infectious disease, comprising administering to a patient an amount of the immunoconjugate of claim 6 or a pharmaceutically acceptable salt or solvate thereof, the amount being effective to treat the infectious disease.

33. An article of manufacture comprising
an antibody drug conjugate compound of claim 6;
a container; and
a package insert or label indicating that the compound can be used to treat cancer characterized by the overexpression of at least one of CD20, CD30, CD33, CD40, CD70 and Lewis Y.

34. A method for the diagnosis of cancer, comprising administering an effective amount of the immunoconjugate of claim 9 to a patient, wherein the immunoconjugate binds to an antigen overexpressed by the cancer; and detecting the immunoconjugate in the patient.

35. A method for the diagnosis of an infectious disease, comprising administering an effective amount of the immunoconjugate of claim 9 to a patient, wherein the immunoconjugate binds to a microbial or viral antigen; and detecting the immunoconjugate in the patient.

36. A method for the diagnosis of an autoimmune disease, comprising administering an effective amount of the immunoconjugate of claim 9 to a patient, wherein the immunoconjugate binds to an antigen associated with the autoimmune disease; and detecting the immunoconjugate in the patient.

37. A method for preparing an immunoconjugate, comprising:
(a) culturing a host cell expressing an engineered antibody, the engineered antibody comprising (i) a functionally active antigen-binding region for a target antigen, (ii) at least one interchain cysteine residue, and (iii) at least one amino acid substitution of an interchain cysteine residue, the host cells being transformed or transfected with an isolated nucleic acid encoding the engineered antibody;
(b) recovering the antibody from the cultured host cells or the culture medium; and
(c) conjugating a diagnostic, preventative or therapeutic agent to the at least one interchain cysteine residue.

38. The method of claim 37, wherein the amino acid substitution is a cysteine to serine substitution.

39. The method of claim 37, wherein the antibody is an intact antibody or an antigen-binding fragment.

40. The method of claim 39, wherein the antigen binding fragment is an Fab, Fab′ or scFvFc.


SEQUENCE LISTING (19 sequences)

SEQ ID NO: 1 - DNA, 42 bp, Artificial
  Primer C226SC229S
gacaaaactcacacatccccaccgtccccagcacctgaactc

SEQ ID NO: 2 - DNA, 38 bp, Artificial
  Primer C220S
gttgagcccaaatcttctgacaaaactcacacatgccc

SEQ ID NO: 3 - DNA, 32 bp, Artificial
  Primer C226S
gacaaaactcacacatccccaccgtgcccagc

SEQ ID NO: 4 - DNA, 38 bp, Artificial
  Primer C220S
gttgagcccaaatcttctgacaaaactcacacatcccc

SEQ ID NO: 5 - DNA, 33 bp, Artificial
  Primer C218S
cttcaacaggggagagtcttagacgcgtattgg

SEQ ID NO: 6 - DNA, 1401 bp, Artificial
  cAC10 H mouse/human chimera
atgggatggagctggatctttcttttcctcctgtcaggaactgcaggtgtccattgtcagatccagctgcagcagtctggacctgaggtggtgaagcctggggcttcagtgaagatatcctgcaaggcttctggctacaccttcactgactactatataacctgggtgaagcagaagcctggacagggacttgagtggattggatggatttatcctggaagcggtaatactaagtacaatgagaagttcaagggcaaggccacattgactgtagacacatcctccagcacagccttcatgcagctcagcagcctgacatctgaggacactgctgtctatttctgtgcgaactatggtaactactggtttgcttactggggccaagggactcaggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatga

SEQ ID NO: 7 - protein, 466 aa, Artificial
  cAC10 H mouse/human chimera
MGWSWIFLFLLSGTAGVHCQIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 717 bp, Artificial
  cAC10 L mouse/human chimera
atggagacagacacaatcctgctatgggtgctgctgctctgggttccaggctccactggtgacattgtgctgacccaatctccagcttctttggctgtgtctctagggcagagggccaccatctcctgcaaggccagccaaagtgttgattttgatggtgatagttatatgaactggtaccaacagaaaccaggacagccacccaaagtcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtggacgttcggtggaggcaccaagctggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 9 - protein, 238 aa, Artificial
  cAC10 L mouse/human chimera
METDTILLWVLLLWVPGSTGDIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 1401 bp, Artificial
  cAC10 H220 cDNA mouse/human chimera
atgggatggagctggatctttcttttcctcctgtcaggaactgcaggtgtccattgtcagatccagctgcagcagtctggacctgaggtggtgaagcctggggcttcagtgaagatatcctgcaaggcttctggctacaccttcactgactactatataacctgggtgaagcagaagcctggacagggacttgagtggattggatggatttatcctggaagcggtaatactaagtacaatgagaagttcaagggcaaggccacattgactgtagacacatcctccagcacagccttcatgcagctcagcagcctgacatctgaggacactgctgtctatttctgtgcgaactatggtaactactggtttgcttactggggccaagggactcaggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatga

SEQ ID NO: 11 - protein, 466 aa, Artificial
  cAC10 H220 mouse/human chimera protein
MGWSWIFLFLLSGTAGVHCQIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 1401 bp, Artificial
  cAC10 H220/226 mouse/human chimera
atgggatggagctggatctttcttttcctcctgtcaggaactgcaggtgtccattgtcagatccagctgcagcagtctggacctgaggtggtgaagcctggggcttcagtgaagatatcctgcaaggcttctggctacaccttcactgactactatataacctgggtgaagcagaagcctggacagggacttgagtggattggatggatttatcctggaagcggtaatactaagtacaatgagaagttcaagggcaaggccacattgactgtagacacatcctccagcacagccttcatgcagctcagcagcctgacatctgaggacactgctgtctatttctgtgcgaactatggtaactactggtttgcttactggggccaagggactcaggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttctgacaaaactcacacatccccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatga

SEQ ID NO: 13 - protein, 466 aa, Artificial
  cAC10 H220/226 mouse/human chimera
MGWSWIFLFLLSGTAGVHCQIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 1401 bp, Artificial
  cAC10 H226/229 mouse/human chimera
atgggatggagctggatctttcttttcctcctgtcaggaactgcaggtgtccattgtcagatccagctgcagcagtctggacctgaggtggtgaagcctggggcttcagtgaagatatcctgcaaggcttctggctacaccttcactgactactatataacctgggtgaagcagaagcctggacagggacttgagtggattggatggatttatcctggaagcggtaatactaagtacaatgagaagttcaagggcaaggccacattgactgtagacacatcctccagcacagccttcatgcagctcagcagcctgacatctgaggacactgctgtctatttctgtgcgaactatggtaactactggtttgcttactggggccaagggactcaggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatccccaccgtccccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatga

SEQ ID NO: 15 - protein, 466 aa, Artificial
  cAC10 H226/229 mouse/human chimera
MGWSWIFLFLLSGTAGVHCQIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 1401 bp, Artificial
  cAC10 H220/226/229 mouse/human chimera cDNA
atgggatggagctggatctttcttttcctcctgtcaggaactgcaggtgtccattgtcagatccagctgcagcagtctggacctgaggtggtgaagcctggggcttcagtgaagatatcctgcaaggcttctggctacaccttcactgactactatataacctgggtgaagcagaagcctggacagggacttgagtggattggatggatttatcctggaagcggtaatactaagtacaatgagaagttcaagggcaaggccacattgactgtagacacatcctccagcacagccttcatgcagctcagcagcctgacatctgaggacactgctgtctatttctgtgcgaactatggtaactactggtttgcttactggggccaagggactcaggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttctgacaaaactcacacatccccaccgtccccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatga

SEQ ID NO: 17 - protein, 466 aa, Artificial
  cAC10 H220/226/229 mouse/human chimera protein
MGWSWIFLFLLSGTAGVHCQIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 717 bp, Artificial
  cAC10 L218 mouse/human chimera cDNA
atggagacagacacaatcctgctatgggtgctgctgctctgggttccaggctccactggtgacattgtgctgacccaatctccagcttctttggctgtgtctctagggcagagggccaccatctcctgcaaggccagccaaagtgttgattttgatggtgatagttatatgaactggtaccaacagaaaccaggacagccacccaaagtcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtggacgttcggtggaggcaccaagctggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtcttag

SEQ ID NO: 19 - protein, 238 aa, Artificial
  cAC10 L218 mouse/human chimera protein
METDTILLWVLLLWVPGSTGDIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES